(19) United States
(12) Patent Application Publication
(10) Pub. No.: US 2022/0173145 A1
(43) Pub. Date: Jun. 2, 2022

(54) IMAGE SENSOR, ENDOSCOPE, AND ENDOSCOPE SYSTEM

(71) Applicant: OLYMPUS CORPORATION, Tokyo (JP)

(72) Inventor: Satoru Adachi, Tokyo (JP)

(73) Assignee: OLYMPUS CORPORATION, Tokyo (JP)

(21) Appl. No.: 17/674,250

(22) Filed: Feb. 17, 2022

Related U.S. Application Data
(63) Continuation of application No. PCT/JP2019/032920, filed on Aug. 22, 2019.

(51) Int. Cl. H01L 27/146; H04N 9/04; H04N 5/3745; H04N 5/225

(52) U.S. Cl. CPC H01L 27/14621; H04N 9/04559; H04N 2005/2255; H04N 5/2256; H04N 5/37455

(57) ABSTRACT

An image sensor includes: a plurality of pixels; a color filter including a plurality of filter units, each filter unit including at least one of a blue filter and a red filter, a green filter, and two or more of special filters; and an imaging controller configured to in a normal observation mode, sequentially output electrical signals generated by the plurality of pixels, and in a special observation mode, sequentially output an additive signal in which electrical signals generated by the plurality of pixels on which at least the two or more of special filters are arranged are added for each of the filter units.

### CROSS-REFERENCE TO RELATED APPLICATION

This application is a continuation of International Application No. PCT/JP2019/032920, filed on Aug. 22, 2019, the entire contents of which are incorporated herein by reference.

### BACKGROUND

### 1. Technical Field

The present disclosure relates to an image sensor that receives light of an object image and generates image data, and relates to an endoscope and an endoscope system.

### 2. Related Art

In an endoscope system, a technology for performing normal observation by radiating continuous light and strobe observation by radiating strobe light at a predetermined timing has been known (for example, Japanese Laid-open Patent Publication No. 2004-97442). In this technology, pulsed strobe light is emitted in synchronization with a frequency of vibration of vocal cords and the vocal cords of a subject that vibrate at a high speed are observed in a stop state or a slow-motion state.

### SUMMARY

In some embodiments, an image sensor includes: a pixel unit including a plurality of pixels that are arranged in a two-dimensional matrix manner, each pixel being configured to perform photoelectric conversion and generate an electrical signal corresponding to intensity of received light; a color filter including a plurality of filter units, each filter unit including at least one of a blue filter and a red filter, a green filter, and two or more of special filters, the plurality of filter units being arranged on the plurality of pixels such that each of filters included in the filter unit corresponds to each of predetermined pixels of the plurality of pixels, the blue filter transmitting light in a wavelength band for blue, the red filter transmitting light in a wavelength band for red, the green filter transmitting light in a wavelength band for green, each of the two or more of special filters being one of a cyan filter and a yellow filter, the cyan filter transmitting light in the wavelength band for blue and light in the wavelength band for green, the yellow filter transmitting light in the wavelength band for green and light in the wavelength band for red; and an imaging controller configured to in a normal observation mode, sequentially output electrical signals generated by the plurality of pixels, and in a special observation mode, sequentially output an additive signal in which electrical signals generated by the plurality of pixels on which at least the two or more of special filters are arranged are added for each of the filter units.

In some embodiments, an endoscope includes: the image sensor; and an insertion portion. The insertion portion includes a distal end portion that is insertable into a subject, and the image sensor is arranged on the distal end portion.

In some embodiments, an endoscope system includes: the endoscope; a light source configured to apply illumination light to the endoscope, the illumination light including at least one of light in the wavelength band for blue and light in the wavelength band for red and including light in the wavelength band for green; and a control device configured to generate a display image based on a digital signal input from the image sensor.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic configuration diagram of an endoscope system according to a first embodiment;

FIG. 2 is a block diagram illustrating a functional configuration of a main part of the endoscope system according to the first embodiment;

FIG. 3 is a diagram illustrating a part of a circuit configuration of a pixel unit;

FIG. 4 is a diagram schematically illustrating arrangement of a color filter;

FIG. 5 is a diagram schematically illustrating sensitivity and a wavelength band of each of the filters;

FIG. 6 is a flowchart illustrating an outline of a process performed by the endoscope system according to the first embodiment;

FIG. 7 is a diagram schematically illustrating read of electrical signals from an image sensor in a normal observation mode;

FIG. 8 is a diagram schematically illustrating pixels added by an imaging control unit;

FIG. 9 is a diagram schematically illustrating read of electrical signals from the image sensor;

FIG. 10 is a diagram schematically illustrating image frames that are output by the image sensor;

FIG. 11 is a comparison diagram for schematically comparing read timings of electrical signals between the normal observation mode and a sensitivity-enhanced observation mode;

FIG. 12 is a diagram schematically illustrating read of electrical signals from the image sensor in a high-speed observation mode;

FIG. 13 is a comparison diagram for schematically comparing read timings of electrical signals between the normal observation mode and the high-speed observation mode;

FIG. 14 is a diagram schematically illustrating pixels added by an imaging control unit according to a first modification of the first embodiment;

FIG. 15 is a diagram schematically illustrating read of electrical signals from an image sensor according to the first modification of the first embodiment;

FIG. 16 is a comparison diagram for schematically comparing read timings of electrical signals between the normal observation mode and in the sensitivity-enhanced observation mode in which three pixels are added;

FIG. 17 is a diagram illustrating a part of a circuit configuration of a pixel unit according to a second embodiment;

FIG. 18 is a diagram schematically illustrating pixels added by an imaging control unit;

FIG. 19 is a diagram schematically illustrating read of electrical signals from an image sensor;

FIG. 20 is a diagram schematically illustrating pixels added by the imaging control unit;

FIG. 21 is a diagram schematically illustrating read of electrical signals from the image sensor;

FIG. 22 is a diagram schematically illustrating pixels added by the imaging control unit;

FIG. 23 is a diagram schematically illustrating read of electrical signals from the image sensor;

FIG. 24 is a diagram schematically illustrating arrangement of a color filter according to a third embodiment;

FIG. 25 is a diagram schematically illustrating sensitivity and a wavelength band of each of filters in the color filter according to the third embodiment;

FIG. 26 is a diagram schematically illustrating pixels added by an imaging control unit in a sensitivity-enhanced observation mode according to the third embodiment;

FIG. 27 is a diagram schematically illustrating read of electrical signals from an image sensor in the sensitivity-enhanced observation mode according to the third embodiment;

FIG. 28 is a diagram schematically illustrating image frames that are output by the image sensor in the sensitivity-enhanced observation mode according to the third embodiment;

FIG. 29 is a diagram schematically illustrating read of electrical signals from the image sensor in a high-speed observation mode according to the third embodiment;

FIG. 30 is a diagram schematically illustrating pixels added by an imaging control unit in a sensitivity-enhanced observation mode according to a modification of the third embodiment;

FIG. 31 is a diagram schematically illustrating read of electrical signals from an image sensor in the sensitivity-enhanced observation mode according to the modification of the third embodiment;

FIG. 32 is a diagram schematically illustrating pixels added by the imaging control unit the sensitivity-enhanced observation mode according to the modification of the third embodiment;

FIG. 33 is a diagram schematically illustrating read of electrical signals from the image sensor in the sensitivity-enhanced observation mode according to the modification of the third embodiment;

FIG. 34 is a diagram schematically illustrating pixels added by the imaging control unit in the sensitivity-enhanced observation mode according to the modification of the third embodiment;

FIG. 35 is a diagram schematically illustrating read of electrical signals from the image sensor in the sensitivity-enhanced observation mode according to the modification of the third embodiment;

FIG. 36 is a diagram schematically illustrating pixels added by the imaging control unit in the sensitivity-enhanced observation mode according to the modification of the third embodiment;

FIG. 37 is a diagram schematically illustrating read of electrical signals from the image sensor in the sensitivity-enhanced observation mode according to the modification of the third embodiment;

FIG. 38 is a diagram schematically illustrating arrangement of a color filter according to a fourth embodiment;

FIG. 39 is a diagram schematically illustrating sensitivity and a wavelength band of each of filters in the color filter according to the fourth embodiment;

FIG. 40 is a diagram schematically illustrating pixels added by an imaging control unit in a sensitivity-enhanced observation mode according to the fourth embodiment;

FIG. 41 is a diagram schematically illustrating read of electrical signals from an image sensor in the sensitivity-enhanced observation mode according to the fourth embodiment;

FIG. 42 is a diagram schematically illustrating image frames that are output by the image sensor in the sensitivity-enhanced observation mode according to the fourth embodiment;

FIG. 43 is a diagram schematically illustrating read of electrical signals from the image sensor in a high-speed observation mode according to the fourth embodiment;

FIG. 44 is a diagram schematically illustrating pixels added by an imaging control unit in a sensitivity-enhanced observation mode according to a modification of the fourth embodiment;

FIG. 45 is a diagram schematically illustrating read of electrical signals from the image sensor in the sensitivity-enhanced observation mode according to the modification of the fourth embodiment;

FIG. 46 is a diagram schematically illustrating pixels added by the imaging control unit in the sensitivity-enhanced observation mode according to the modification of the fourth embodiment;

FIG. 47 is a diagram schematically illustrating read of electrical signals from the image sensor in the sensitivity-enhanced observation mode according to the modification of the fourth embodiment;

FIG. 48 is a diagram schematically illustrating pixels added by the imaging control unit in the sensitivity-enhanced observation mode according to the modification of the fourth embodiment;

FIG. 49 is a diagram schematically illustrating read of electrical signals from the image sensor in the sensitivity-enhanced observation mode according to the modification of the fourth embodiment;

FIG. 50 is a diagram schematically illustrating pixels added by the imaging control unit in the sensitivity-enhanced observation mode according to the modification of the fourth embodiment;

FIG. 51 is a diagram schematically illustrating read of electrical signals from the image sensor in the sensitivity-enhanced observation mode according to the modification of the fourth embodiment;

FIG. 52 is a diagram schematically illustrating sensitivity and a wavelength band of each of filters in a color filter according to other embodiments; and

FIG. 53 is a diagram schematically illustrating sensitivity and a wavelength band of each of filters in a color filter according to other embodiments.

### DETAILED DESCRIPTION

Embodiments for carrying out the present disclosure will be described in detail below with reference to the drawings. The present disclosure is not limited by the embodiments below. In addition, in the drawings referred to in the following description, shapes, sizes, and positional relationships are only schematically illustrated so that the content of the present disclosure may be understood. In other words, the present disclosure is not limited to only the shapes, the sizes, and the positional relationships illustrated in the drawings.

### First Embodiment

Configuration of Endoscope System

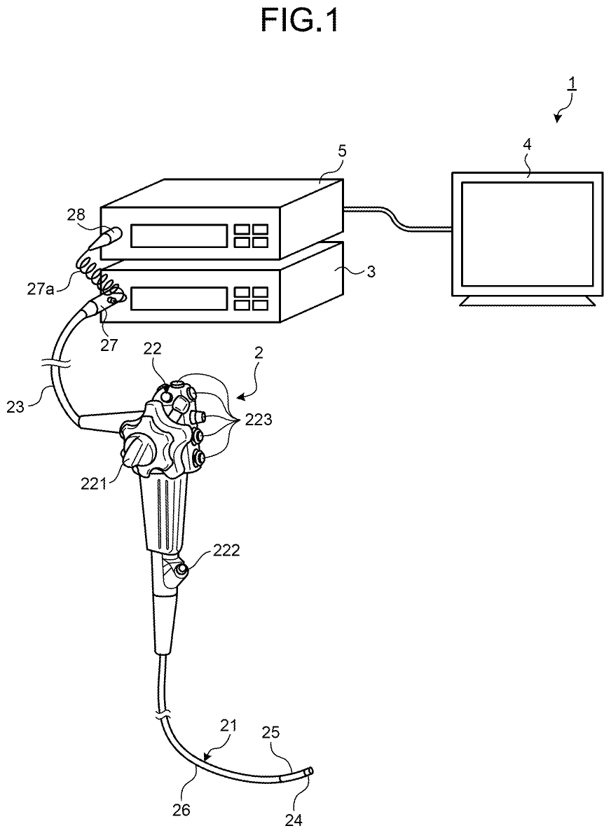

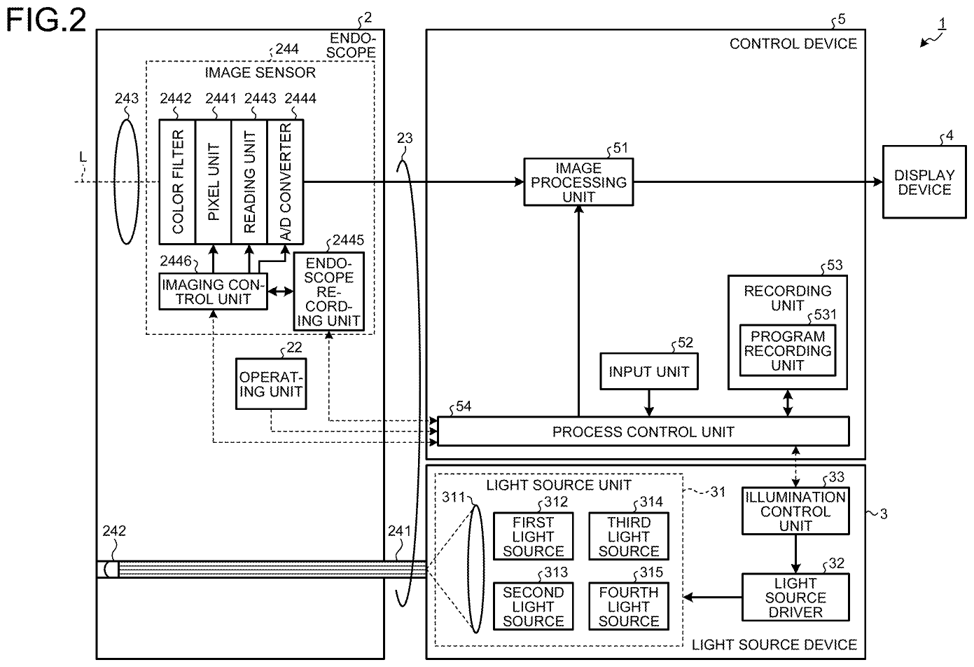

FIG. 1 is a schematic configuration diagram of an endoscope system according to a first embodiment. FIG. 2 is a block diagram illustrating a functional configuration of a main part of the endoscope system according to the first embodiment.

An endoscope system 1 illustrated in FIG. 1 and FIG. 2 inserts an endoscope into a subject, such as patient, captures an image of an inside of a body or vocal cords of the subject, and displays a display image based on the captured image data on a display device. A user, such as a doctor, observes the display image displayed on the display device, and examines whether each of a bleeding site, a tumor site, and an abnormal site that are examination target sites are present. The endoscope system 1 includes an endoscope 2, a light source device 3, a display device 4, and a control device 5 (processor).

Configuration of Endoscope

A configuration of the endoscope 2 will be described below.

The endoscope 2 generates image data (RAW data) in which an image of the inside of the body or the vocal cords of the subject is captured, and outputs the generated image data to the control device 5. The endoscope 2 includes an insertion portion 21, an operating unit 22, and a universal cord 23.

The insertion portion 21 has a flexible thin and elongated shape. The insertion portion 21 includes a distal end portion 24 in which an image sensor 244 (to be described later) is incorporated, a bending portion 25 that is constructed with a plurality of bending pieces and that is freely bendable, and a flexible tube portion 26 that is connected to a proximal end side of the bending portion 25 and that has a flexible elongated shape.

The distal end portion 24 is configured with fiberglass or the like. The distal end portion 24 includes a light guide 241 that serves as an optical waveguide for light supplied from the light source device 3, an illumination lens 242 that is arranged on a distal end of the light guide 241, an optical system 243 for collecting light, and the image sensor 244 that is arranged at an image forming position of the optical system 243.

The image sensor 244 includes a plurality of pixels that are arranged in a two-dimensional manner. Each of the pixels performs photoelectric conversion and generates an electrical signal corresponding to intensity of received light that is collected by the optical system 243. The image sensor 244 is configured with an image sensor, such as a complementary metal oxide semiconductor (CMOS). Specifically, the image sensor 244 includes a plurality of pixels that are arranged in a two-dimensional manner, where the pixels receive light, perform photoelectric conversion, and output electrical signals. The image sensor 244 captures an image of an object (body cavity) at a predetermined frame rate and outputs image data (RAW data). The image sensor 244 includes a pixel unit 2441 that serves as a pixel portion, a color filter 2442, a reading unit 2443, an analog-to-digital (A/D) converter 2444, an endoscope recording unit 2445, and an imaging control unit 2446.

The pixel unit 2441 includes a plurality of pixels that are arranged in a two-dimensional matrix manner. Each of the pixels performs photoelectric conversion, generates an electrical signal corresponding to intensity of received light, and outputs the electrical signal.

Circuit Configuration of Pixel Unit

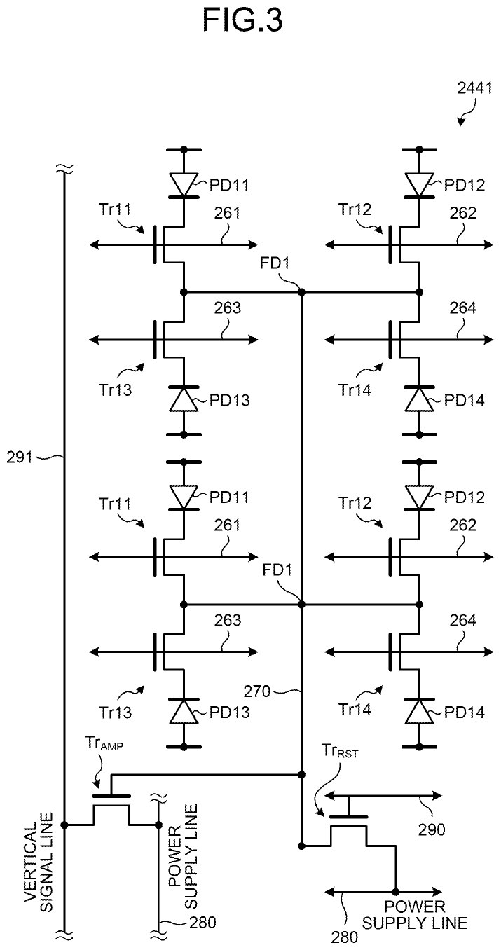

A circuit configuration of the pixel unit 2441 will be described in detail below. FIG. 3 is a diagram illustrating a part of the circuit configuration of the pixel unit 2441. Meanwhile, in FIG. 3, for simplicity of explanation, four pixels (2×2) are adopted as a minimum pixel unit of the pixel unit 2441.

As illustrated in FIG. 3, the pixel unit 2441 causes four pixels (2×2) to output electrical signals via a single charge-voltage converter FD1. The pixel unit 2441 includes four photoelectric conversion elements PD (PD11, PD12, PD13, and PD14), the charge-voltage converter FD1, four transfer transistors Tr (Tr11, Tr12, Tr13, and Tr14), a charge-voltage conversion reset transistor Tr_{RST}, and a pixel output transistor Tr_{AMP}. Meanwhile, in the first embodiment, the four photoelectric conversion elements PD (PD11, PD12, PD13, and PD14) and the transfer transistors Tr (Tr11, Tr12, Tr13, and Tr14) for transferring signal charges from the respective photoelectric conversion elements PD to the charge-voltage converter FD1 are referred to as unit pixels (unit pixels of 2×2).

The photoelectric conversion element PD11 to the photoelectric conversion element PD14 perform photoelectric conversion on incident light to obtain signal charge amounts corresponding to intensity of the incident light, and accumulate the signal charge amounts. Cathode sides of the photoelectric conversion element PD11 to the photoelectric conversion element PD14 are respectively connected to source sides of the transfer transistor Tr11 to the transfer transistor Tr14, and anode sides of the photoelectric conversion element PD11 to the photoelectric conversion element PD14 are connected to ground GND.

The transfer transistor Tr11 to the transfer transistor Tr14 respectively transfer charges from the photoelectric conversion element PD11 to the photoelectric conversion element PD14 to the charge-voltage converter FD1. Drains of the transfer transistor Tr11 to the transfer transistor Tr14 are connected to a source of the charge-voltage conversion reset transistor Tr_{RST}. Further, gates of the transfer transistor Tr11 to the transfer transistor Tr14 are respectively connected to a signal line 261 to a signal line 264 to each of which a driving pulse for reading an independent row is applied.

The charge-voltage converter FD1 is configured with floating diffusion, and converts charges accumulated in the photoelectric conversion element PD11 to the photoelectric conversion element PD14 into voltage. The charge-voltage converter FD1 is connected to a gate of the pixel output transistor Tr_{AMP} via a signal line 270.

A drain of the charge-voltage conversion reset transistor Tr_{RST} is connected to a power supply line 280, and a gate of the charge-voltage conversion reset transistor Tr_{RST} is connected to a reset line 290 to which a reset pulse is applied. The charge-voltage conversion reset transistor Tr_{RST} resets the charge-voltage converter FD1 at a predetermined potential.

A source of the pixel output transistor Tr_{AMP} is connected to a vertical signal line 291, and a drain of the pixel output transistor Tr_{AMP} is connected to the power supply line 280. The pixel output transistor Tr_{AMP} outputs an electrical signal that is converted to voltage by the charge-voltage converter FD1 to the vertical signal line 291. The pixel output transistor Tr_{AMP} enters an ON state when the charge-voltage conversion reset transistor Tr_{RST} resets the charge-voltage converter FD1 at predetermined voltage, and outputs the electrical signal that is converted to voltage by the charge-voltage converter FD1 to the vertical signal line 291.

The pixel unit 2441 configured as described above transfers charges accumulated in the photoelectric conversion element PD11 to the photoelectric conversion element PD14 to the charge-voltage converter FD1 via the transfer transistor Tr11 to the transfer transistor Tr14 under the control of the imaging control unit 2446 (to be described later). Then, the electrical signal converted by the charge-voltage converter FD1 is input to the gate of the pixel output transistor Tr_{AMP} via the signal line 270, is amplified, and is output to the vertical signal line 291. Thereafter, the charge-voltage converter FD1 is reset at the predetermined potential by the charge-voltage conversion reset transistor Tr_{RST}, so that the pixel output transistor Tr_{AMP} enters an OFF state.

Configuration of Color Filter

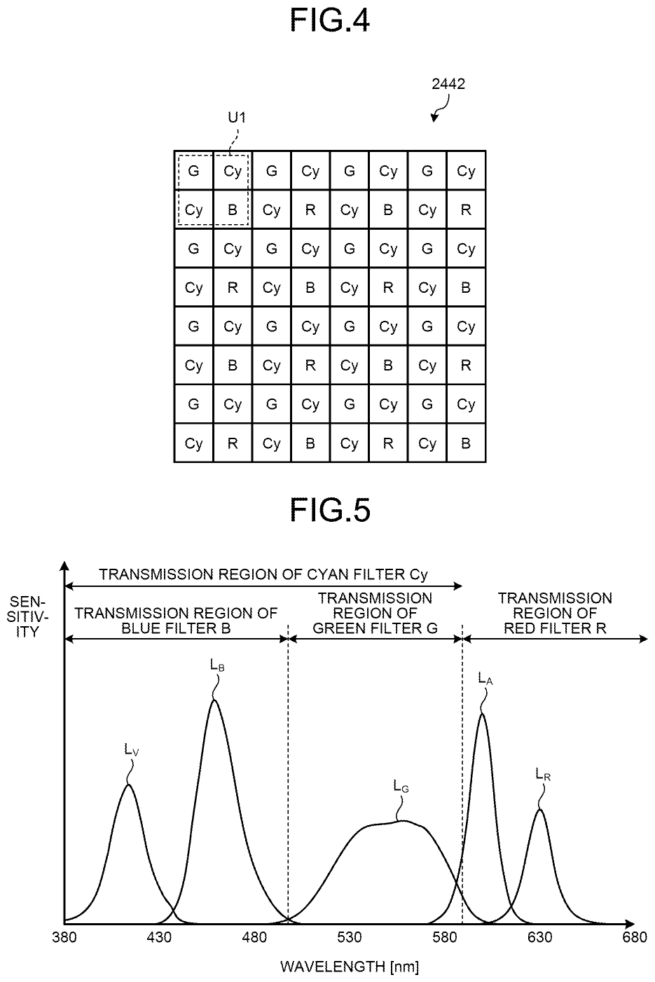

The color filter 2442 will be described in detail below. FIG. 4 is a diagram schematically illustrating arrangement of the color filter 2442.

As illustrated in FIG. 4, the unit pixels (2×2) constitute a single filter unit U1, and filters are arranged on light receiving surfaces of the photoelectric conversion element PD11 to the photoelectric conversion element PD14. The filter unit U1 is configured with at least one of a blue filter B and a red filter R, a green filter G, and two or more special filters. The blue filter B transmits light in a wavelength band for blue. The red filter R transmits light in a wavelength band for red. The green filter G transmits light in a wavelength band for green. The special filter is configured with a cyan filter Cy. The cyan filter Cy transmits at least two or more of light in the wavelength band for blue and light in the wavelength band for green.

FIG. 5 is a diagram schematically illustrating sensitivity and a wavelength band of each of the filters. In FIG. 5, a horizontal axis represents a wavelength (nm) and a vertical axis represents sensitivity. Further, in FIG. 5, a curve L_{V} represents a wavelength band for purple, a curve L_{E} represents the wavelength band for blue, a curve L_{G} represents the wavelength band for green, a curve L_{A} represents a wavelength band for umber, and a curve L_{R} represents the wavelength band for red.

As illustrated in FIG. 5, the cyan filter Cy transmits light in the wavelength band for blue and light in the wavelength band for green. Meanwhile, in the description below, the photoelectric conversion element PD in which the red filter R is arranged on the light receiving surface is referred to as an R pixel, the photoelectric conversion element PD in which the green filter G is arranged on the light receiving surface is referred to as a G pixel, the photoelectric conversion element PD in which the blue filter B is arranged on the light receiving surface is referred to as a B pixel, and the photoelectric conversion element PD in which the cyan filter Cy is arranged is referred to as a Cy pixel.

Referring back to FIG. 2, explanation of the image sensor 244 is continued.

The reading unit 2443 applies a driving pulse to the transfer transistors Tr11 to Tr14 and transfers charges from the photoelectric conversion elements PD11 to PD14 to the charge-voltage converter FD1 under the control of the imaging control unit 2446. Subsequently, the reading unit 2443 supplies power supply voltage to the pixel output transistor Tr_{AMP} and outputs an electrical signal that is converted to voltage by the charge-voltage converter FD1 to the vertical signal line 291 under the control of the imaging control unit 2446. Then, the reading unit 2443 applies a reset pulse to the charge-voltage conversion reset transistor Tr_{RST} and resets the charge-voltage converter FD1 at the predetermined potential under the control of the imaging control unit 2446. The reading unit 2443 is configured with a vertical scanning circuit, a horizontal scanning circuit, and the like.

The A/D converter 2444 converts an analog electrical signal input from the reading unit 2443 to a predetermined-bit digital electrical signal, and outputs the digital electrical signal under the control of the imaging control unit 2446. For example, the A/D converter 2444 performs conversion to 10-bit digital electrical signal and outputs the digital electrical signal to outside. The A/D converter 2444 is configured with an A/D conversion circuit or the like.

The endoscope recording unit 2445 records therein various kinds of information on the endoscope 2. For example, the endoscope recording unit 2445 records therein identification information for identifying the endoscope 2, identification information on the image sensor 244, and the like. The endoscope recording unit 2445 is configured with a non-volatile memory or the like.

The imaging control unit 2446 controls operation of the image sensor 244 on the basis of instruction information input from the control device 5. Specifically, the imaging control unit 2446 controls a frame rate and an imaging timing of the image sensor 244 on the basis of the instruction information input from the control device 5. More specifically, if an instruction signal for designating a normal observation mode is input from the control device 5, the imaging control unit 2446 sequentially outputs electrical signals generated by all of the photoelectric conversion elements PD. In contrast, if an instruction signal for designating a special observation mode is input from the control device 5, the imaging control unit 2446 adds electrical signals generated by the plurality of Cy pixels for each of the filter units U1, and outputs the additive signal to outside. For example, the imaging control unit 2446 causes the reading unit 2443 to apply a driving pulse to the transfer transistor Tr12 and the transfer transistor Tr13 such that charges are transferred from the photoelectric conversion element PD12 and the photoelectric conversion element PD13 to the charge-voltage converter FD1 so that signal charges are added. Then, the imaging control unit 2446 causes the reading unit 2443 to transfer an additive signal in which the electrical signals of the plurality of Cy pixels are added by the charge-voltage converter FD1 to the vertical signal line 291. The imaging control unit 2446 is configured with a timing generator or the like.

The operating unit 22 includes a bending knob 221 that causes the bending portion 25 to bend in a vertical direction and in a horizontal direction, a treatment tool insertion portion 222 for inserting a treatment tool, such as a biopsy forceps, a laser scalpel, and an inspection probe, into a body cavity, and a plurality of switches 223 that are operation input units for inputting an operation instruction signal for the light source device 3, the control device 5, and peripheral devices, such as an air supply means, a water supply means, and a gas supply means, and for inputting a pre-freeze signal for instructing the image sensor 244 to capture a still image. The treatment tool inserted from the treatment tool insertion portion 222 gets out of an aperture (not illustrated) via a treatment tool channel (not illustrated) of the distal end portion 24.

The universal cord 23 includes, inside thereof, at least the light guide 241 and an assembly cable as a collection of one or more cables. The assembly cable is signal lines for transmitting and receiving signals between the endoscope 2, the light source device 3, and the control device 5, and includes a signal line for transmitting and receiving setting data, a signal line for transmitting and receiving image data, a signal line for transmitting and receiving a driving timing signal for driving the image sensor 244, and the like. The universal cord 23 includes a connector 27 that is detachably attached to the light source device 3. The connector 27 includes a coil cable 27a that has a coil shape and that is extended, and a connector 28 that is arranged at an extended end of the coil cable 27a and that is detachably attached to the control device 5.

Configuration of Light Source Device

A configuration of the light source device 3 will be described below.

The light source device 3 supplies illumination light for illuminating a subject from the distal end portion 24 of the endoscope 2. The light source device 3 includes a light source unit 31, a light source driver 32, and an illumination control unit 33.

The light source unit 31 applies illumination light that includes at least one of light in the wavelength band for red and light in the wavelength band for blue and that includes light in the wavelength band for green or applies special light that includes light in the wavelength band for green and narrow band light (for example, in a wavelength band of 415 nm+540 nm). The light source unit 31 includes a condenser lens 311, a first light source 312, a second light source 313, a third light source 314, and a fourth light source 315.

The condenser lens 311 is configured with one or more lenses. The condenser lens 311 collects light emitted by the first light source 312, the second light source 313, the third light source 314, and the fourth light source 315 and outputs the collected light to the light guide 241.

The first light source 312 is configured with a light emitting diode (LED) lamp for red. The first light source 312 emits light in the wavelength band for red (hereinafter, simply referred to as “R light”) on the basis of an electric current supplied from the light source driver 32.

The second light source 313 is configured with an LED lamp for green. The second light source 313 emits light in the wavelength band for green (hereinafter, simply referred to as “G light”) on the basis of an electric current supplied from the light source driver 32.

The third light source 314 is configured with an LED lamp for blue. The third light source 314 emits light in the wavelength band for blue (hereinafter, simply referred to as “B light”) on the basis of an electric current supplied from the light source driver 32.

The fourth light source 315 is configured with an LED lamp for purple. The fourth light source 315 emits light in the wavelength band for purple (for example, 415 nm±10) on the basis of an electric current supplied from the light source driver 32.

The light source driver 32 supplies electric currents to the first light source 312, the second light source 313, the third light source 314, and the fourth light source 315 under the control of the illumination control unit 33, and emits light that corresponds to an observation mode set in the endoscope system 1. Specifically, if the observation mode set in the endoscope system 1 is the normal observation mode, the light source driver 32 causes the first light source 312, the second light source 313, and the third light source 314 to emit light to emit white light, under the control of the illumination control unit 33 (simultaneous method). Further, if the observation mode set in the endoscope system 1 is a special light observation mode, the light source driver 32 causes the second light source 313 and the fourth light source 315 to emit light to emit narrow band light, under the control of the illumination control unit 33.

The illumination control unit 33 controls lighting timing of the light source device 3 on the basis of an instruction signal received from the control device 5. Specifically, the illumination control unit 33 causes the first light source 312, the second light source 313, and the third light source 314 to emit light with certain cycles. The illumination control unit 33 is configured with a central processing unit (CPU) or the like. Further, if the observation mode of the endoscope system 1 is the special light observation mode, the illumination control unit 33 controls the light source driver 32 such that narrow band light is emitted by combination of the second light source 313 and the fourth light source 315. Meanwhile, the illumination control unit 33 may control the light source driver 32 in accordance with the observation mode of the endoscope system 1, and cause a combination of two or more of the first light source 312, the second light source 313, the third light source 314, and the fourth light source 315 to emit light.

Configuration of Display Device

A configuration of the display device 4 will be described below.

The display device 4 displays an image corresponding to image data that is generated by the endoscope 2 and that is received from the control device 5. The display device 4 displays various kinds of information on the endoscope system 1. The display device 4 is configured with a display panel made of liquid crystal, organic electro luminescence (EL), or the like.

Configuration of Control Device

A configuration of the control device 5 will be described below.

The control device 5 receives image data generated by the endoscope 2, performs predetermined image processing on the received image data, and outputs the image data to the display device 4. Further, the control device 5 comprehensively controls entire operation of the endoscope system 1. The control device 5 includes an image processing unit 51, an input unit 52, a recording unit 53, and a process control unit 54.

The image processing unit 51 receives image data generated by the endoscope 2, performs predetermined image processing on the received image data, and outputs the image data to the display device 4, under the control of the process control unit 54. The image processing unit 51 is configured with a processor including a memory and hardware, such as a graphics processing unit (GPU), a digital signal processor (DSP), or a field programmable gate array (FPGA).

The input unit 52 receives input of an instruction signal for designating operation of the endoscope system 1, and outputs the received instruction signal to the process control unit 54. For example, the input unit 52 receives input of an instruction signal for designating the normal observation mode or the special observation mode, and outputs the received instruction signal to the process control unit 54. The input unit 52 is configured with a switch, a button, a touch panel, or the like. Here, the normal observation mode is a mode in which the image sensor 244 outputs an electrical signal that is output from each of an R pixel, a G pixel, a B pixel, and a Cy pixel. Further, the special observation mode includes a sensitivity-enhanced observation mode and a high-speed observation mode. The sensitivity-enhanced observation mode is a mode in which electrical signals that are generated by at least the plurality of Cy pixels are added, and an additive signal that is obtained by the addition is output from the image sensor 244. The high-speed observation mode is a mode in which electrical signals that are generated by at least the plurality of Cy pixels are added, an additive signal that is obtained by the addition is output from the image sensor 244, and charges in the R pixel, the G pixel, and the B pixel are reset without reading electrical signals from the R pixel, the G pixel, and the B pixel.

The recording unit 53 records therein various programs to be executed by the endoscope system 1, data being executed by the endoscope system 1, and image data generated by the endoscope 2. The recording unit 53 is configured with a volatile memory, a non-volatile memory, a memory card, and the like. The recording unit 53 includes a program recording unit 531 that records therein various programs to be executed by the endoscope system 1.

The process control unit 54 is configured with a processor including a memory and hardware, such as an FPGA or a CPU. The process control unit 54 controls each of the units of the endoscope system 1. For example, if an instruction signal for changing illumination light emitted by the light source device 3 is input from the input unit 52, the process control unit 54 controls the illumination control unit 33 and changes the illumination light emitted by the light source device 3.

Process Performed by Endoscope System

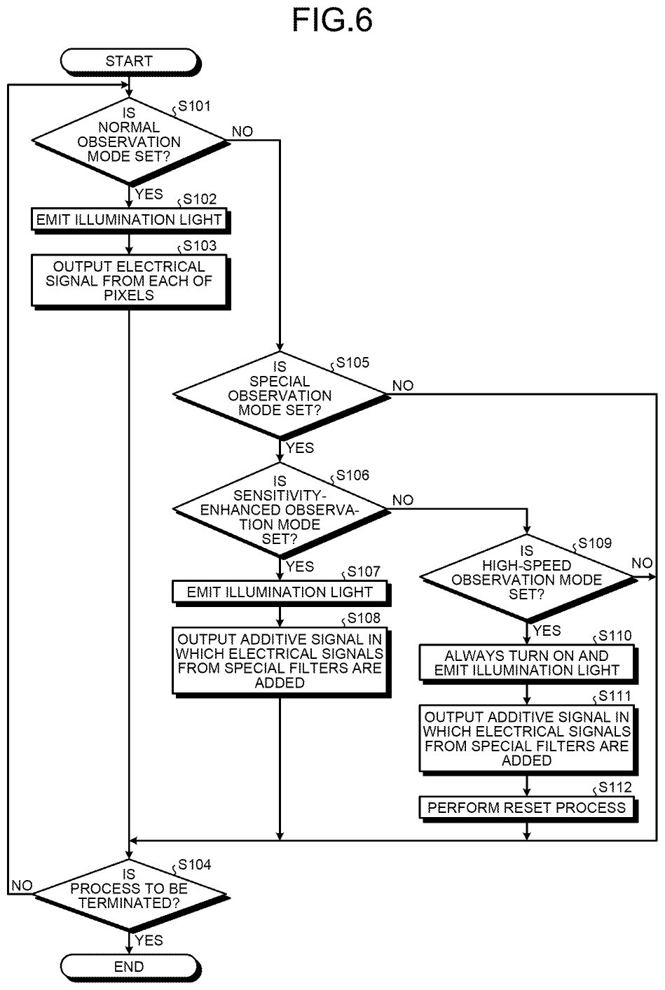

A process performed by the endoscope system 1 will be described below. FIG. 6 is a flowchart illustrating an outline of the process performed by the endoscope system 1.

As illustrated in FIG. 6, first, a case will be described in which the endoscope system 1 is set to the normal observation mode (Step S101: Yes). In this case, the process control unit 54 causes the light source device 3 to emit illumination light in a vertical blanking interval (Step S102).

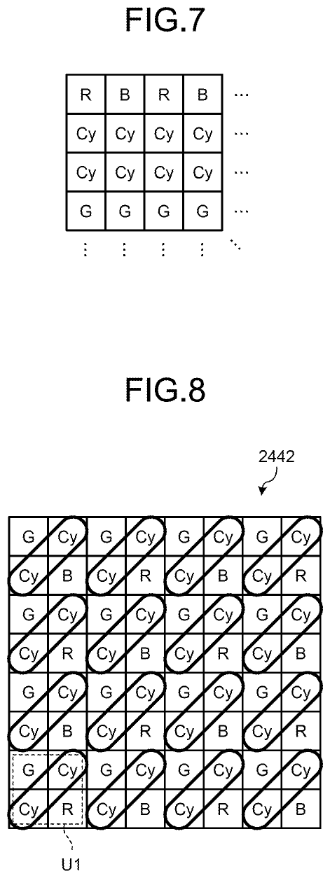

Subsequently, the imaging control unit 2446 sequentially outputs electrical signals generated by the plurality of pixels of the pixel unit 2441 (Step S103). Specifically, as illustrated in FIG. 7, the imaging control unit 2446 causes the reading unit 2443 to sequentially outputs electrical signals generated by the plurality of pixels.

Thereafter, if an instruction signal for designating termination is input from the input unit 52 (Step S104: Yes), the endoscope system 1 terminates the process. In contrast, if the instruction signal designating termination is not input from the input unit 52 (Step S104: No), the endoscope system 1 returns to Step S101 as described above.

A case will be described in which the endoscope system 1 is not set to the normal observation mode at Step S101 (Step S101: No). In this case, if the endoscope system 1 is set to the special observation mode (Step S105: Yes), the endoscope system 1 proceeds to Step S106 to be described later. In contrast, if the endoscope system 1 is not set to the special observation mode (Step S105: No), the endoscope system 1 proceeds to Step S104.

A case will be described in which the endoscope system 1 is set to the sensitivity-enhanced observation mode at Step S106 (Step S106: Yes). In this case, the process control unit 54 causes the light source device 3 to emit illumination light in the vertical blanking interval (Step S107).

Subsequently, the imaging control unit 2446 outputs an additive signal in which the electrical signals generated by the plurality of Cy pixels on which the special filters are arranged are added for each of the filter units U1 to outside (Step S108). After Step S108, the endoscope system 1 proceeds to Step S104.

Method of Reading Electrical Signals

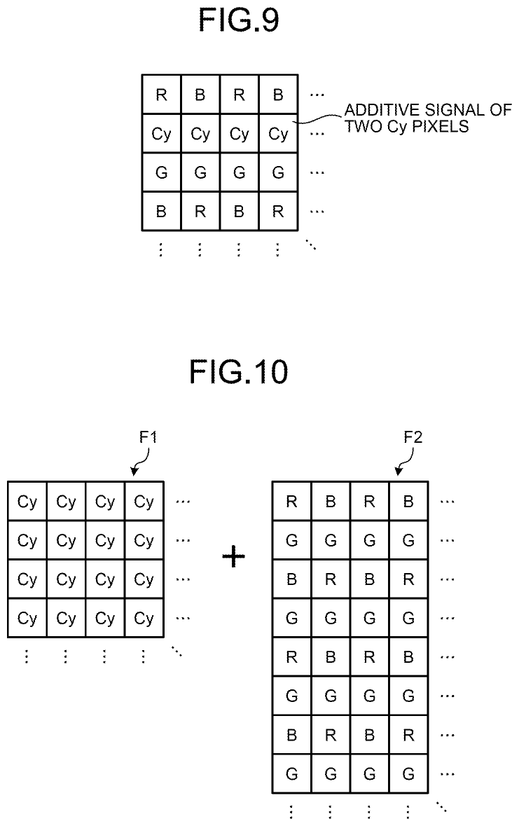

A method of reading electrical signals by the imaging control unit 2446 will be described below. FIG. 8 is a diagram schematically illustrating pixels added by the imaging control unit 2446. FIG. 9 is a diagram schematically illustrating read of electrical signals from the image sensor 244. FIG. 10 is a diagram schematically illustrating image frames that are output from the image sensor 244.

As illustrated in FIG. 8 and FIG. 9, the imaging control unit 2446 causes the reading unit 2443 to apply a driving pulse to the transfer transistor Tr12 and the transfer transistor Tr13, so that charges are transferred from the photoelectric conversion element PD12 and the photoelectric conversion element PD13 in which the cyan filters Cy are arranged on the light receiving surfaces to the charge-voltage converter FD1. Then, the imaging control unit 2446 causes the reading unit 2443 to output an additive signal that is obtained by addition performed by the charge-voltage converter FD1, for each of the filter units U1. Further, as illustrated in FIG. 10, the imaging control unit 2446 causes the pixel unit 2441 to alternately output a pixel mixed frame F1 in which the electrical signals generated by the plurality of Cy pixels are added, and a pixel non-mixed frame F2 that is generated by each of the R pixel, the G pixel, and the B pixel. As a result, because of wide wavelength transmission regions of the Cy pixels, the image sensor 244 is able to achieve sensitivity that is about four times higher than those of the R pixel, the G pixel, and the B pixel by adding the electrical signals of the two Cy pixels. Furthermore, the image sensor 244 reads and outputs the electrical signals from the R pixel, the G pixel, and the B pixel without adding the electrical signals, so that it is possible to increase a dynamic range by four times (12 dB).

Read Timings

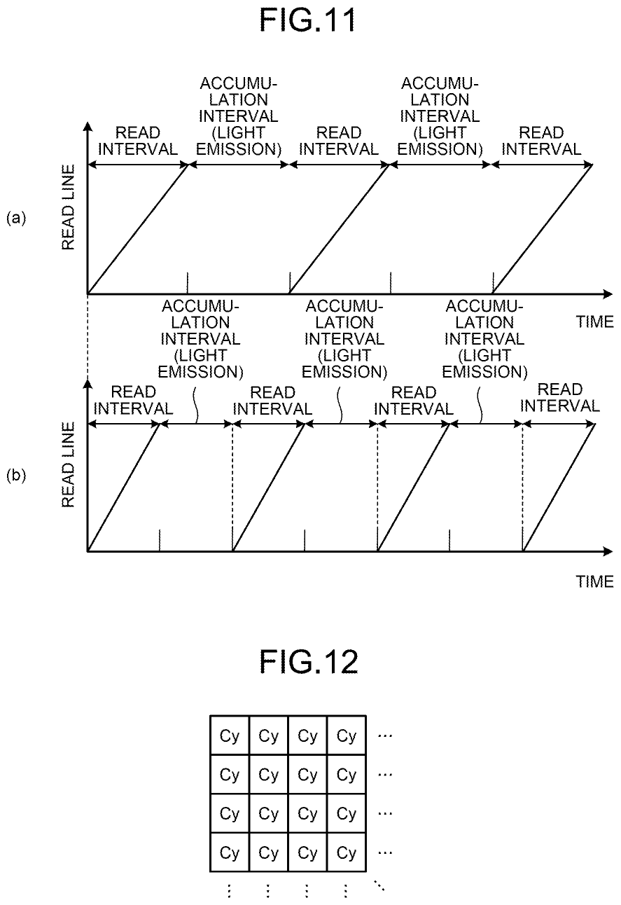

Read timings of electrical signals in the normal observation mode and the sensitivity-enhanced observation mode will be described below. FIG. 11 is a comparison diagram for schematically comparing read timings of electrical signals between the normal observation mode and the sensitivity-enhanced observation mode. In FIG. 11, (a) in an upper stage illustrates a read timing in the normal observation mode, and (b) in a lower stage illustrates a read timing in the sensitivity-enhanced observation mode.

As illustrated in FIG. 11, with use of the sensitivity-enhanced observation mode, it is possible to increase sensitivity due to pixel mixture, so that it is possible to reduce an accumulation time as compared to the normal observation mode. Further, with use of the sensitivity-enhanced observation mode, it is possible to reduce the number of pixels to be read due to pixel mixture, so that it is possible to reduce a read interval as compared to the normal observation mode.

Referring back to FIG. 6, explanation is continued.

A case will be described in which the endoscope system 1 is set to the sensitivity-enhanced observation mode at Step S106 (Step S106: No). In this case, the endoscope system 1 proceeds to Step S109.

At Step S109, if the endoscope system 1 is set to the high-speed observation mode (Step S109: Yes), the endoscope system 1 proceeds to Step S110. In contrast, if the endoscope system 1 is not set to the high-speed observation mode (Step S109: No), the endoscope system 1 proceeds to Step S104.

At Step S110, the process control unit 54 causes the light source device 3 to always turn on and emit illumination light independently of the vertical blanking (Step S110).

Subsequently, the imaging control unit 2446 adds, for each of the filter units U1, the electrical signals generated by the plurality of pixels on which the cyan filters Cy are arranged, outputs the additive signal to outside (Step S111), and resets the pixels other than the plurality of pixels on which the cyan filters Cy are arranged (Step S112). After Step S112, the endoscope system 1 proceeds to Step S104.

Method of Reading Electrical Signals

A method of reading electrical signals from the image sensor 244 in the high-speed observation mode will be described below. FIG. 12 is a diagram schematically illustrating read of electrical signals from the image sensor 244 in the high-speed observation mode.

As illustrated in FIG. 12, the imaging control unit 2446 causes the reading unit 2443 to apply a driving pulse to the transfer transistor Tr12 and the transfer transistor Tr13, so that charges are transferred from the photoelectric conversion element PD12 and the photoelectric conversion element PD13 to the charge-voltage converter FD1. Then, the imaging control unit 2446 causes the reading unit 2443 to output an additive signal that is obtained by addition performed by the charge-voltage converter FD1, for each of the filter units U1. In this case, the imaging control unit 2446 resets the electrical signals of the pixels other than the plurality of Cy pixels, that is, the electrical signals of the R pixel, the G pixel, and the B pixel, without reading the electrical signals.

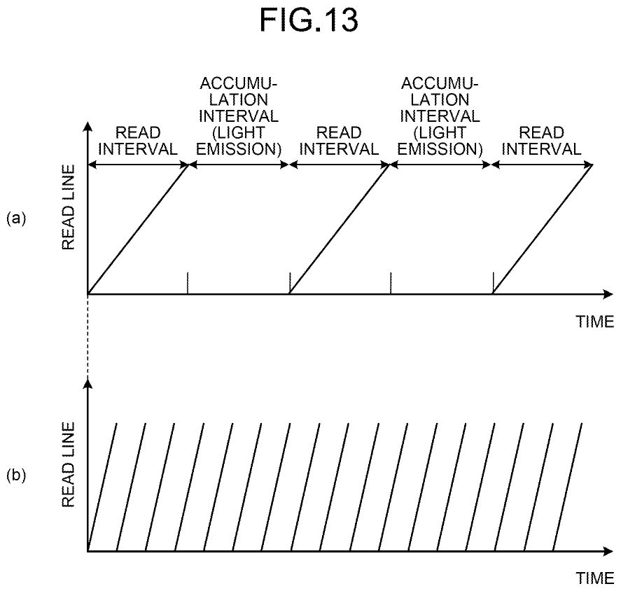

FIG. 13 is a comparison diagram for schematically comparing read timings of electrical signals between the normal observation mode and the high-speed observation mode. In FIG. 13, (a) in an upper stage illustrates a read timing in the normal observation mode, and (b) in a lower stage illustrates a read timing in the high-speed observation mode.

As illustrated in FIG. 13, with use of the high-speed observation mode, it is possible to increase sensitivity due to pixel mixture, so that it is possible to reduce an accumulation time as compared to the normal observation mode. Further, with use of the high-speed observation mode, it is possible to reduce the number of pixels to be read due to pixel mixture, so that it is possible to reduce a read interval as compared to the normal observation mode. Furthermore, by performing discharge without outputting the electrical signals from the R pixel, the G pixel, and the B pixel, read times for reading the R pixel, the G pixel, and the B pixel are not needed, so that it is possible to further reduce the read interval as compared to the sensitivity-enhanced observation mode.

According to the first embodiment as described above, in the special observation mode, the imaging control unit 2446 adds, for each of the filter units U1, the electrical signals generated by the plurality of Cy pixels on which the special filters are arranged, and sequentially outputs the additive signals to outside. Therefore, it is possible to increase sensitivity due to pixel mixture and reduce an accumulation time as compared to the normal observation mode. As a result, it is possible to perform imaging at high speed and it is possible to further reduce a size of the image sensor 244.

Furthermore, according to the first embodiment, in the high-speed observation mode, the imaging control unit 2446 performs discharge without outputting the electrical signals from the R pixel, the G pixel, and the B pixel and resets the R pixel, the G pixel, and the B pixel, so that it is possible to further reduce the read interval as compared to the sensitivity-enhanced observation mode.

Moreover, according to the first embodiment, because of wide wavelength transmission regions of the Cy pixels, the imaging control unit 2446 is able to achieve sensitivity that is about four times higher than those of the R pixel, the G pixel, and the B pixel by adding the electrical signals of the two Cy pixels, and reads and outputs the electrical signals from the R pixel, the G pixel, and the B pixel without adding the electrical signals, so that it is possible to increase a dynamic range by four times (12 dB).

Furthermore, according to the first embodiment, in the special observation mode, the imaging control unit 2446 adds, for each of the filter units U1, the electrical signals generated by the plurality of Cy pixels on which the special filters are arranged, sequentially outputs the additive signals to outside, and sequentially outputs the electrical signals generated by the R pixel, the G pixel, and the B pixel. Therefore, it is possible to increase sensitivity due to synthesis with a non-mixed electrical signal, and simultaneously ensure color resolution.

Moreover, according to the first embodiment, in the special observation mode, the imaging control unit 2446 adds, for each of the filter units U1, the electrical signals generated by the plurality of Cy pixels on which the special filters are arranged, and sequentially outputs the additive signals to outside. Therefore, it becomes not necessary to perform imaging twice for long-time exposure and short-time exposure to increase sensitivity, and it is possible to prevent generation of an artifact due to motion.

### First Modification of First Embodiment

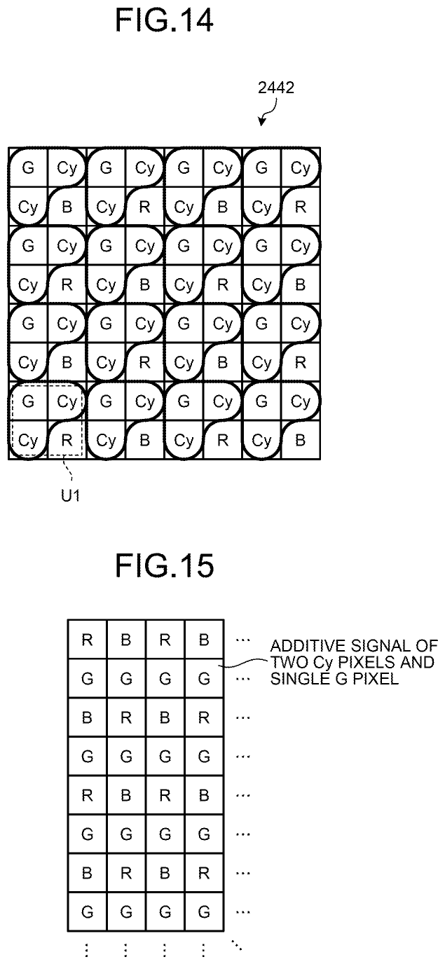

A first modification of the first embodiment will be described below. FIG. 14 is a diagram schematically illustrating pixels added by an imaging control unit according to the first modification of the first embodiment. FIG. 15 is a diagram schematically illustrating read of electrical signals from an image sensor according to the first modification of the first embodiment.

As illustrated in FIG. 14 and FIG. 15, the imaging control unit 2446 adds, for each of the filter units U1, electrical signals of the two Cy pixels and an electrical signal of the G pixel, and outputs an additive signal to outside. Specifically, the imaging control unit 2446 causes the reading unit 2443 to apply a driving pulse to the transfer transistor Tr11, the transfer transistor Tr12, and the transfer transistor Tr13. Then, the imaging control unit 2446 transfers charges from the photoelectric conversion element PD11, in which the green filter G is arranged on the light receiving surface, and the photoelectric conversion element PD12 and the photoelectric conversion element PD13, in each of which the cyan filter Cy is arranged on the light receiving surface, to the charge-voltage converter FD1. Thereafter, the imaging control unit 2446 causes the reading unit 2443 to output an additive signal that is obtained by addition performed by the charge-voltage converter FD1, for each of the filter units U1.

Read Timings

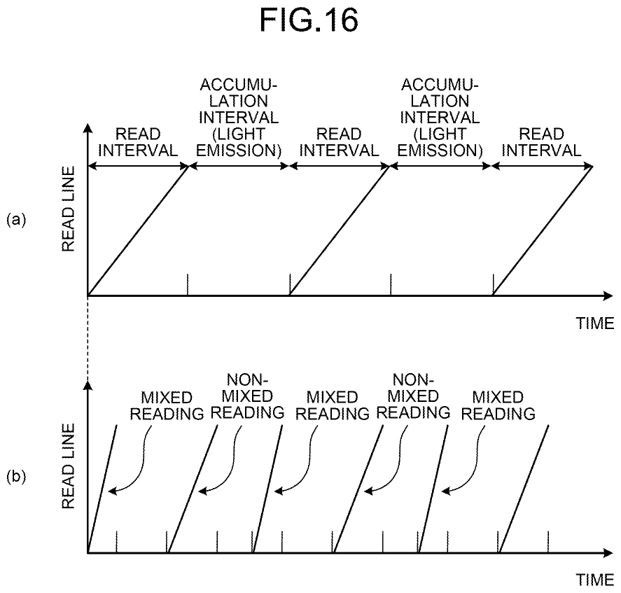

Read timings of electrical signals in the normal observation mode and the sensitivity-enhanced observation mode will be described below. FIG. 16 is a comparison diagram for schematically comparing read timings of electrical signals in the normal observation mode and in the sensitivity-enhanced observation mode in which the three pixels are added. In FIG. 16, (a) in an upper stage illustrates a read timing in the normal observation mode, and (b) in a lower stage illustrates a read timing of the sensitivity-enhanced observation mode in which the three pixels are added.

As illustrated in FIG. 16, with use of the sensitivity-enhanced observation mode in which the three pixels are added, it is possible to increase sensitivity due to pixel mixture, so that it is possible to reduce an accumulation time as compared to the normal observation mode. Further, with use of the sensitivity-enhanced observation mode, it is possible to reduce the number of pixels to be read due to pixel mixture, so that it is possible to reduce a read interval as compared to the normal observation mode.

According to the first modification of the first embodiment, in the special observation mode, the imaging control unit 2446 adds, for each of the filter units U1, at least the electrical signals generated by the plurality of Cy pixels and the electrical signal generated by the G pixel, and outputs an additive signal to outside. Therefore, it is possible to reduce the number of pixels to be read due to pixel mixture and it is possible to reduce an accumulation time as compared to the normal observation mode. As a result, it is possible to perform imaging at high speed and further reduce the size of the image sensor 244.

Meanwhile, in the first modification of the first embodiment, similarly to the first embodiment as described above, it may be possible to reset the electrical signal of the B pixel and the electrical signal of the R pixel without reading the electrical signals.

Furthermore, in the first modification of the first embodiment, the imaging control unit 2446 may add the electrical signals of the two Cy pixels, the G pixel, the R pixel, and the B pixel of the pixel unit 2441, and cause the pixel unit 2441 to output an additive signal obtained by the addition. With this configuration, it is possible to reduce the read interval and achieve high sensitivity.

### Second Embodiment

A second embodiment will be described. In the first embodiment as described above, pixels of 2×2 are adopted as the unit pixels, but in the second embodiment, pixels of 2×4 are adopted as the unit pixels. Therefore, in the following, a configuration of a pixel unit according to the second embodiment will be described, and thereafter, a method of reading signals by an imaging control unit will be described. Meanwhile, the same components as those of the endoscope system 1 according to the first embodiment as described above are denoted by the same reference symbols, and detailed explanation thereof will be omitted.

Circuit Configuration of Pixel Unit

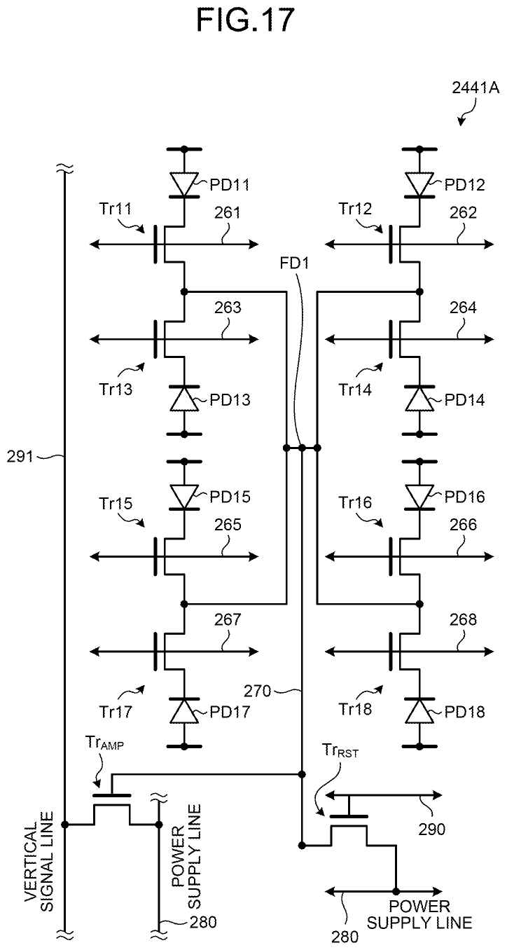

FIG. 17 is a diagram illustrating a part of a circuit configuration of the pixel unit according to the second embodiment. Meanwhile, in FIG. 17, for simplicity of explanation, eight pixels (4×2) are adopted as a minimum pixel unit of a pixel unit 2441A. Further, in the second embodiment, the color filter 2442 of the first embodiment as described above is arranged on a light receiving surface of each of the photoelectric conversion element PD11 to a photoelectric conversion element PD18.

As illustrated in FIG. 17, the pixel unit 2441A causes eight pixels (4×2) to output electrical signals via a single charge-voltage converter. The pixel unit 2441 includes the eight photoelectric conversion elements PD (PD11 to PD18), the charge-voltage converter FD1, eight transfer transistors Tr (Tr11 to Tr18), the charge-voltage conversion reset transistor Tr_{RST}, and the pixel output transistor Tr_{AMP}. Further, in the second embodiment, the eight photoelectric conversion elements PD (PD11 to PD18) and the transfer transistors Tr (Tr11 to Tr18) for transferring signal charges from the respective photoelectric conversion elements PD to the charge-voltage converter FD1 are referred to as unit pixels (unit pixels of 4×2).

The photoelectric conversion element PD11 to the photoelectric conversion element PD18 perform photoelectric conversion on incident light to obtain signal charge amounts corresponding to intensity of the incident light, and accumulate the signal charge amounts. Cathode sides of the photoelectric conversion element PD11 to the photoelectric conversion element PD18 are respectively connected to source sides of the transfer transistor Tr11 to the transfer transistor Tr18, and anode sides of the photoelectric conversion element PD11 to the photoelectric conversion element PD18 are connected to ground GND.

The transfer transistor Tr11 to the transfer transistor Tr18 respectively transfer charges from the photoelectric conversion element PD11 to the photoelectric conversion element PD18 to the charge-voltage converter FD1. Drains of the transfer transistor Tr11 to the transfer transistor Tr18 are connected to the source of the charge-voltage conversion reset transistor Tr_{RST}. Further, gates of the transfer transistor Tr11 to the transfer transistor Tr18 are respectively connected to the signal line 261 to the signal line 264 to each of which a driving pulse for reading an independent row is applied.

The charge-voltage converter FD1 is configured with floating diffusion, and converts charges accumulated in the photoelectric conversion element PD11 to the photoelectric conversion element PD18 into voltage. The charge-voltage converter FD1 is connected to the gate of the pixel output transistor Tr_{AMP} via the signal line 270.

The drain of the charge-voltage conversion reset transistor Tr_{RST} is connected to the power supply line 280, and the gate of the charge-voltage conversion reset transistor Tr_{RST} is connected to the reset line 290 to which a reset pulse is applied. The charge-voltage conversion reset transistor Tr_{RST} resets the charge-voltage converter FD1 at a predetermined potential.

The source of the pixel output transistor Tr_{AMP} is connected to the vertical signal line 291, and the drain of the pixel output transistor Tr_{AMP} is connected to the power supply line 280. The pixel output transistor Tr_{AMP} outputs an electrical signal that is converted to voltage by the charge-voltage converter FD1 to the vertical signal line 291. The pixel output transistor Tr_{AMP} enters the ON state when the charge-voltage conversion reset transistor Tr_{MT} resets the charge-voltage converter FD1 at predetermined voltage, and outputs the electrical signal that is converted to voltage by the charge-voltage converter FD1 to the vertical signal line 291.

The pixel unit 2441A configured as described above transfers charges accumulated in the photoelectric conversion element PD11 to the photoelectric conversion element PD18 to the charge-voltage converter FD1 via the transfer transistor Tr11 to the transfer transistor Tr18 under the control of the imaging control unit 2446. Then, the electrical signal converted by the charge-voltage converter FD1 is input to the gate of the pixel output transistor Tr_{AMP} via the signal line 270, is amplified, and is output to the vertical signal line 291.

Method of Reading Electrical Signals

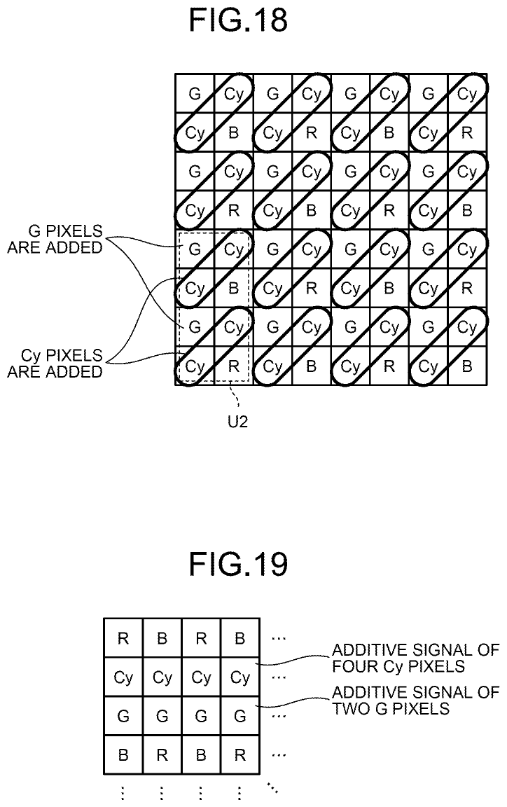

A method of reading electrical signals by the pixel unit 2441A will be described below. FIG. 18 is a diagram schematically illustrating pixels added by the imaging control unit 2446. FIG. 19 is a diagram schematically illustrating read of electrical signals from an image sensor 244A.

As illustrated in FIG. 18 and FIG. 19, the imaging control unit 2446 causes the reading unit 2443 to apply a driving pulse to the transfer transistor Tr12, the transfer transistor Tr13, the transfer transistor Tr16, and the transfer transistor Tr17, so that charges are transferred from the four photoelectric conversion elements, that is, the photoelectric conversion element PD12, the photoelectric conversion element PD13, the photoelectric conversion element PD16, and the photoelectric conversion element PD17, to the charge-voltage converter FD1. Further, the imaging control unit 2446 causes the reading unit 2443 to output an additive signal in which the electrical signals of the four Cy pixels are added by the charge-voltage converter FD1, for each of filter units U2.

Furthermore, the imaging control unit 2446 causes the reading unit 2443 to apply a driving pulse to the transfer transistor Tr11 and the transfer transistor Tr15, so that charges are transferred from two photoelectric conversion elements, that is, the photoelectric conversion element PD11 and the photoelectric conversion element PD15, to the charge-voltage converter FD1. Then, the imaging control unit 2446 causes the reading unit 2443 to output an additive signal in which the electrical signals of the two G pixels are added by the charge-voltage converter FD1, for each of the filter units U2. Meanwhile, the imaging control unit 2446 may reset the electrical signals of the pixels other than the plurality of pixels on which the cyan filter Cy are arranged, that is, the electrical signals of the blue filter B, the red filter R, and the green filter G, without reading the electrical signals.

According to the second embodiment as described above, the imaging control unit 2446 outputs, for each of the filter units U2, an additive signal in which the electrical signals generated by the four Cy pixels are added and an additive signal in which the electrical signals generated by the two G pixels are added. Therefore, it is possible to reduce an accumulation time as compared to the normal observation mode. As a result, it is possible to achieve high sensitivity, perform imaging at high speed, and further reduce the size of the image sensor 244.

Meanwhile, in the second embodiment, the imaging control unit 2446 may reset the electrical signals of the pixels other than the plurality of Cy pixels, that is, the electrical signals of the R pixel, the G pixel, and the B pixel, without reading the electrical signals. With this configuration, it is possible to further increase imaging speed.

In the second embodiment, pixels of 2×4 are adopted as the unit pixels, and the unit pixels in which two pixels are arranged in the horizontal direction and four pixels are arranged in the vertical direction are described: however, pixels of 4×2, in which four pixels are arranged in the horizontal direction and two pixels are arranged in the vertical direction, may be adopted as the unit pixels.

### First Modification of Second Embodiment

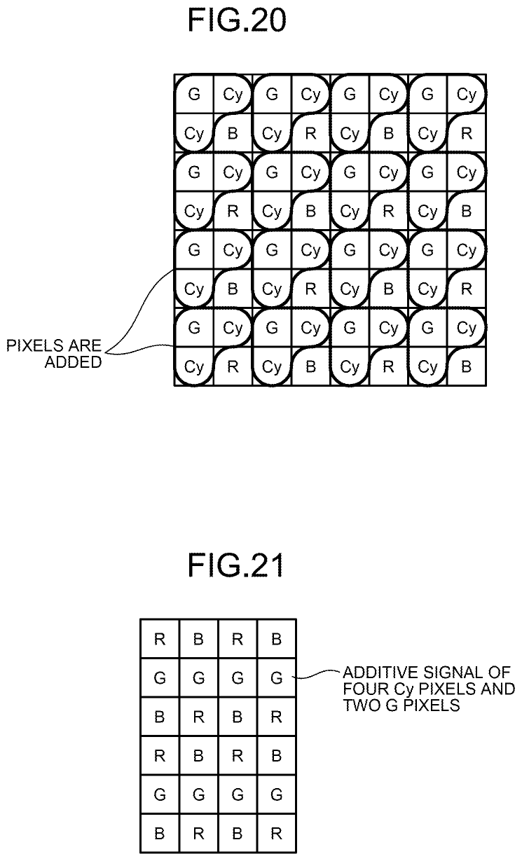

A first modification of the second embodiment will be described below. FIG. 20 is a diagram schematically illustrating pixels added by the imaging control unit 2446. FIG. 21 is a diagram schematically illustrating read of electrical signals from the image sensor 244A.

As illustrated in FIG. 20 and FIG. 21, the imaging control unit 2446 causes the reading unit 2443 to apply a driving pulse to the transfer transistor Tr11, the transfer transistor Tr12, the transfer transistor Tr13, the transfer transistor Tr15, the transfer transistor Tr16, and the transfer transistor Tr17, so that charges are transferred from the six photoelectric conversion elements, that is, the photoelectric conversion element PD11, the photoelectric conversion element PD12, the photoelectric conversion element PD13, the photoelectric conversion element PD15, the photoelectric conversion element PD16, and the photoelectric conversion element PD17, to the charge-voltage converter FD1. Then, the imaging control unit 2446 causes the reading unit 2443 to output an additive signal in which the electrical signals of the four Cy pixels and the electrical signals of two G pixels are added by the charge-voltage converter FD1, for each of the filter units U2.

According to the first modification of the second embodiment as described above, the imaging control unit 2446 outputs an additive signal in which the electrical signals of the four Cy pixels and the electrical signals of two G pixels are added by the charge-voltage converter FD1 for each of the filter units U2. Therefore, it is possible to reduce an accumulation time as compared to the normal observation mode. As a result, it is possible to achieve high sensitivity, perform imaging at high speed, and further reduce the size of the image sensor 244.

Meanwhile, in the first modification of the second embodiment, the imaging control unit 2446 may reset the electrical signals of the pixels other than the plurality of Cy pixels and the plurality of G pixels, that is, the electrical signals of the R pixel and the B pixel, without reading the electrical signals. With this configuration, it is possible to further increase imaging speed.

### Second Modification of Second Embodiment

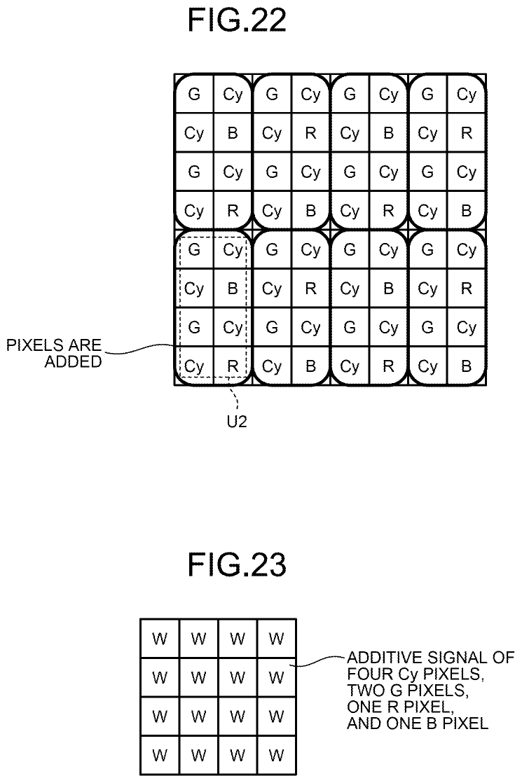

A second modification of the second embodiment will be described below. FIG. 22 is a diagram schematically illustrating pixels added by the imaging control unit 2446. FIG. 23 is a diagram schematically illustrating read of electrical signals from the image sensor 244A.

As illustrated in FIG. 22 and FIG. 23, the imaging control unit 2446 causes the reading unit 2443 to apply a driving pulse to the transfer transistor Tr11 to the transfer transistor Tr18, so that charges are transferred from the eight photoelectric conversion elements, that is, the photoelectric conversion element PD11 to the photoelectric conversion element PD18, to the charge-voltage converter FD1. Then, the imaging control unit 2446 causes the reading unit 2443 to output an additive signal in which the electrical signals of the four Cy pixels, the electrical signals of the two G pixels, the electrical signal of the R pixel, and the electrical signal of the B pixel are added by the charge-voltage converter FD1, for each of the filter units U2.

According to the second modification of the second embodiment as described above, the imaging control unit 2446 outputs an additive signal in which the electrical signals of all of the pixels in the filter unit U2 are added for each of the filter units U2, so that it is possible to further increase imaging speed and achieve high sensitivity.

### Third Embodiment

A third embodiment will be described below. In the first and the second embodiments as described above, the filters Cy are used as the special filters and included in the filter unit of the color filter: however, in the third embodiment, a filter Ye that transmits light in the wavelength band for red and light in the wavelength band for green is adopted as the special filter of the color filter. In the following, a configuration of the color filter according to the third embodiment is described, and thereafter, a method of reading an electrical signal from each of the pixels will be described. Meanwhile, the same components as those of the endoscope system 1 according to the first embodiment as described above are denoted by the same reference symbols, and detailed explanation thereof will be omitted.

Configuration of Color Filter

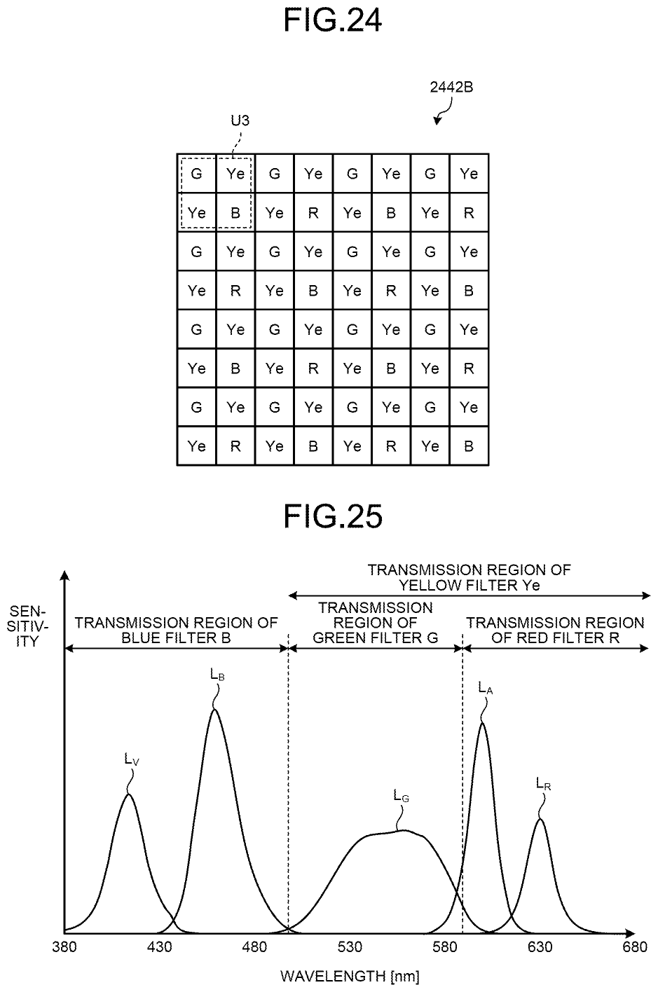

A color filter according to the third embodiment will be described in detail below. FIG. 24 is a diagram schematically illustrating arrangement of the color filter according to the third embodiment.

A color filter 2442B illustrated in FIG. 24 is configured such that unit pixels (2×2) constitute a single filter unit U3, and filters are arranged on the light receiving surfaces of the photoelectric conversion element PD11 to the photoelectric conversion element PD14. The filter unit U3 is configured with at least one of the blue filter B and the red filter R, the green filter G, and two or more special filters. The special filter is configured with the yellow filter Ye. The yellow filter Ye transmits light in the wavelength band for red and light in the wavelength band for green.

FIG. 25 is a diagram schematically illustrating sensitivity and a wavelength band of each of the filters. In FIG. 25, a horizontal axis represents a wavelength (nm) and a vertical axis represents sensitivity. Further, in FIG. 25, a curve L_{V} represents a wavelength band for purple, a curve L_{B} represents a wavelength band for blue, a curve L_{G} represents a wavelength band for green, a curve L_{A} represents a wavelength band for umber, and a curve L_{R} represents a wavelength band for red.

As illustrated in FIG. 25, the yellow filter Ye transmits light in the wavelength band for red and light in the wavelength band for green. Further, in the following description, the photoelectric conversion element PD on which the yellow filter Ye is arranged is referred to as a Ye pixel.

Method of Reading Electrical Signals

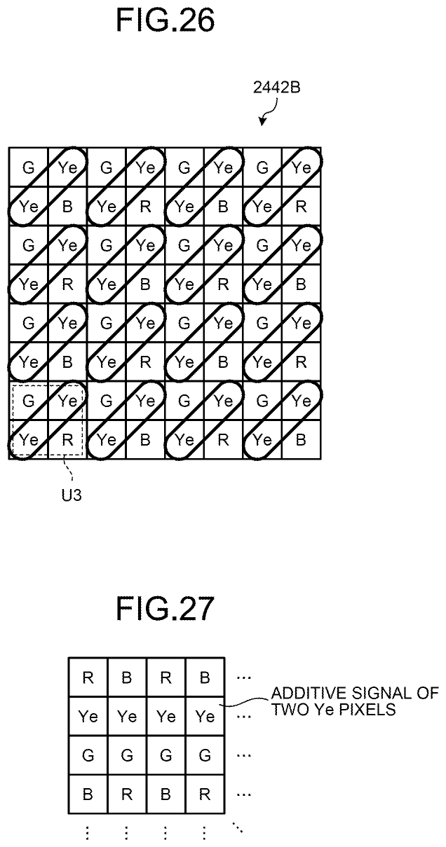

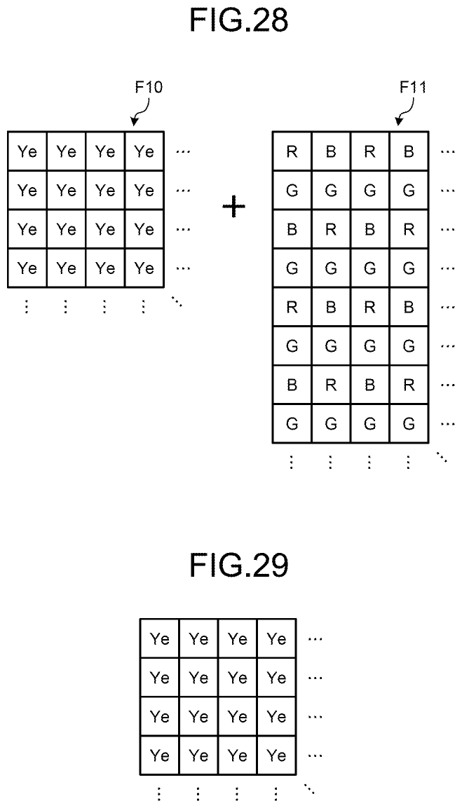

A method of reading electrical signals by the imaging control unit 2446 in the sensitivity-enhanced observation mode will be described below. FIG. 26 is a diagram schematically illustrating pixels added by the imaging control unit 2446 in the sensitivity-enhanced observation mode. FIG. 27 is a diagram schematically illustrating read of electrical signals from the image sensor 244 in the sensitivity-enhanced observation mode. FIG. 28 is a diagram schematically illustrating image frames that are output by the image sensor 244 in the sensitivity-enhanced observation mode.

As illustrated in FIG. 26 and FIG. 27, the imaging control unit 2446 causes the reading unit 2443 to apply a driving pulse to the transfer transistor Tr12 and the transfer transistor Tr13, so that charges are transferred from the photoelectric conversion element PD12 and the photoelectric conversion element PD13, in each of which the yellow filter Ye is arranged on the light receiving surface, to the charge-voltage converter FD1. Then, the imaging control unit 2446 causes the reading unit 2443 to output an additive signal that is obtained by addition performed by the charge-voltage converter FD1, for each of the filter units U3. Further, as illustrated in FIG. 28, the imaging control unit 2446 causes the pixel unit 2441 to alternately output a pixel mixed frame F10 in which the electrical signals generated by the plurality of Ye pixels are added, and a pixel non-mixed frame F11 that is generated by each of the R pixel, the G pixel, and the B pixel. As a result, because of wide wavelength transmission regions of the Ye pixels, the image sensor 244 is able to achieve sensitivity that is about four times higher than those of the R pixel, the G pixel, and the B pixel by adding the electrical signals of the two Ye pixels. Furthermore, the image sensor 244 reads and outputs the electrical signals from the R pixel, the G pixel, and the B pixel without adding the electrical signals, so that it is possible to increase a dynamic range by four times (12 dB). Meanwhile, a read timing in the sensitivity-enhanced observation mode is the same as that of the first embodiment as described above, and therefore, detailed explanation thereof will be omitted.

A method of reading electrical signals from the image sensor 244 in the high-speed observation mode will be described below. FIG. 29 is a diagram schematically illustrating read of electrical signals from the image sensor 244 in the high-speed observation mode.

As illustrated in FIG. 29, the imaging control unit 2446 causes the reading unit 2443 to apply a driving pulse to the transfer transistor Tr12 and the transfer transistor Tr13, so that charges are transferred from the photoelectric conversion element PD12 and the photoelectric conversion element PD13 to the charge-voltage converter FD1. Then, the imaging control unit 2446 causes the reading unit 2443 to output an additive signal that is obtained by addition performed by the charge-voltage converter FD1, for each of the filter units U3. In this case, the imaging control unit 2446 resets the electrical signals of the pixels other than the plurality of Ye pixels, that is, the electrical signals of the R pixel, the G pixel, and the B pixel without reading the electrical signals. Meanwhile, a read timing in the high-speed observation mode is the same as that of the first embodiment as described above, and therefore, detailed explanation thereof will be omitted.

According to the third embodiment as described above, it is possible to reduce an accumulation time as compared to the normal observation, so that it is possible to perform imaging at high speed and it is possible to further reduce the size of the image sensor 244.

Furthermore, according to the third embodiment, in the special observation mode, the imaging control unit 2446 adds, for each of the filter units U3, the electrical signals generated by the plurality of Ye pixels on which the special filters are arranged, and sequentially outputs the additive signals to outside. Therefore, it is possible to increase sensitivity due to pixel mixture and reduce an accumulation time as compared to the normal observation mode. As a result, it is possible to perform imaging at high speed and it is possible to further reduce the size of the image sensor 244.

Moreover, according to the third embodiment, in the high-speed observation mode, the imaging control unit 2446 performs discharges without outputting the electrical signals from the R pixel, the G pixel, and the B pixel and resets the R pixel, the G pixel, and the B pixel, so that it is possible to further reduce a read interval as compared to the sensitivity-enhanced observation mode.

Furthermore, according to the third embodiment, because of wide wavelength transmission regions of the Ye pixels, the imaging control unit 2446 is able to achieve sensitivity that is about four times higher than those of the R pixel, the G pixel, and the B pixel by adding the electrical signals of the two Ye pixels, and reads and outputs the electrical signals from the R pixel, the G pixel, and the B pixel without adding the electrical signals, so that it is possible to increase a dynamic range by four times (12 dB).

Moreover, according to the third embodiment, in the special observation mode, the imaging control unit 2446 adds, for each of the filter units U3, the electrical signals generated by the plurality of Ye pixels on which the special filters are arranged, sequentially outputs the additive signals to outside, and sequentially outputs the electrical signals generated by the R pixel, the G pixel, and the B pixel. Therefore, it is possible to increase sensitivity due to synthesis with a non-mixed electrical signal, and simultaneously ensure color resolution.

Furthermore, according to the third embodiment, in the special observation mode the imaging control unit 2446 adds, for each of the filter units U3, the electrical signals generated by the plurality of Ye pixels on which the special filters are arranged, and sequentially outputs the additive signals to outside. Therefore, it becomes not necessary to perform imaging twice for long-time exposure and short-time exposure to increase sensitivity, and it is possible to prevent generation of an artifact due to motion.

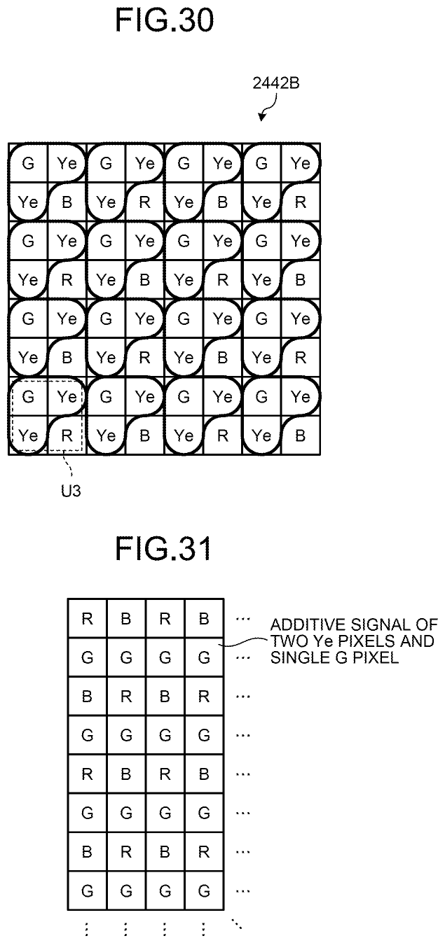

Meanwhile, in the third embodiment, similarly to the first modification of the first embodiment as described above, as illustrated in FIG. 30 and FIG. 31, the imaging control unit 2446 may control the reading unit 2443 and perform the same process as that of the first modification of the first embodiment as described above such that the electrical signals of the two Ye pixels and the electrical signal of the G pixel are added and output to outside for each of the filter units U3.

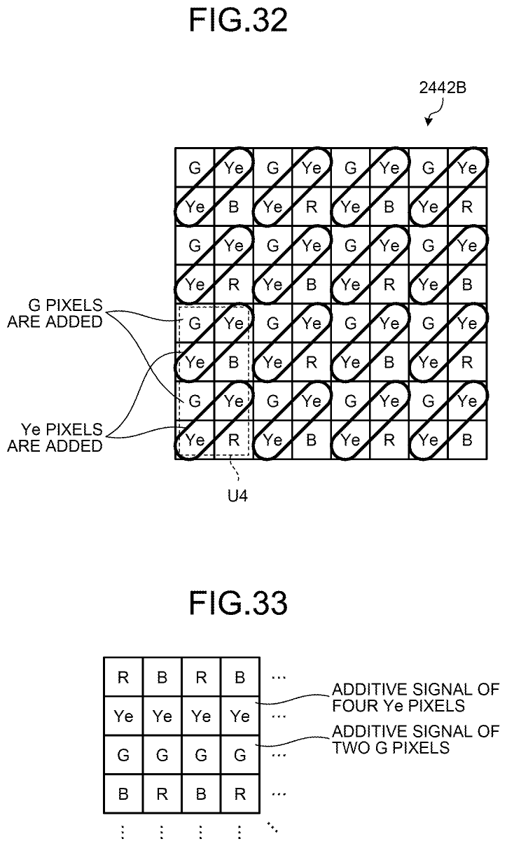

Furthermore, in the third embodiment, if the pixel unit 2441A of the second embodiment as described above is used, as illustrated in FIG. 32 and FIG. 33, the imaging control unit 2446 may control the reading unit 2443 and preform the same process as that of the second embodiment as described above to output an additive signal in which the electrical signals of the four Ye pixels are added for each of filter units U4.

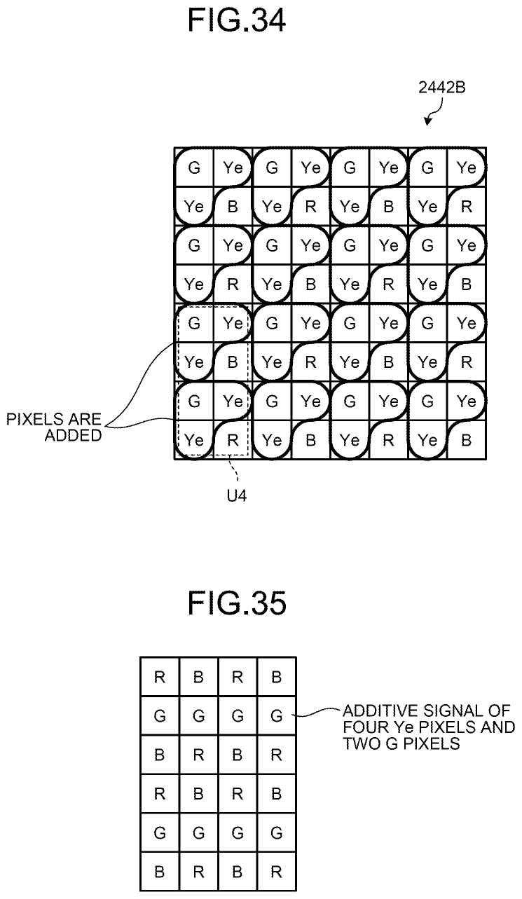

Moreover, in the third embodiment, if the pixel unit 2441A of the second embodiment as described above is used, as illustrated in FIG. 34 and FIG. 35, the imaging control unit 2446 may control the reading unit 2443 and perform the same process as that of the first modification of the second embodiment as described above to output an additive signal in which the electrical signals of the four Ye pixels and the electrical signals of the two G pixels are added by the charge-voltage converter FD1 for each of the filter units U4.

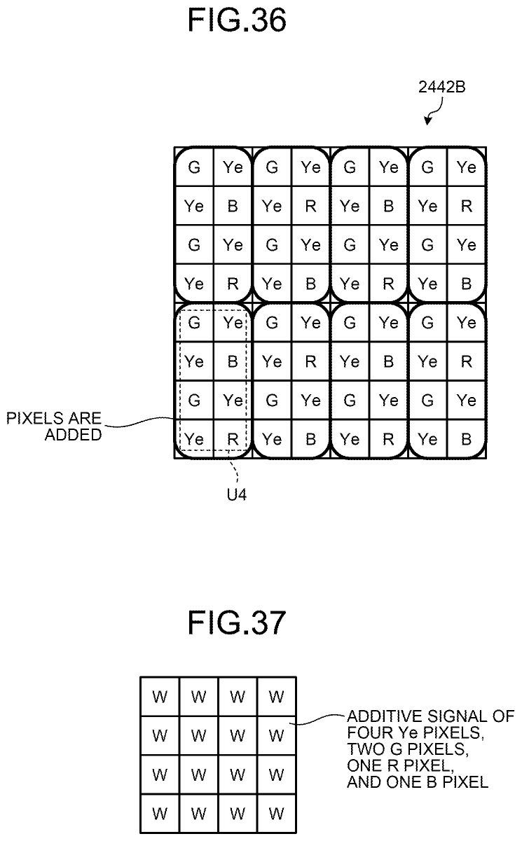

Furthermore, in the third embodiment, if the pixel unit 2441A of the second embodiment as described above is used, as illustrated in FIG. 36 and FIG. 37, the imaging control unit 2446 may control the reading unit 2443 and perform the same process as that of the second modification of the second embodiment as described above to output an additive signal in which the electrical signals of the four Ye pixels, the electrical signals of the two G pixels, the electrical signal of the R pixel, and the electrical signal of the B pixel are added by the charge-voltage converter FDL for each of the filter units U4.

### Fourth Embodiment

A fourth embodiment will be described below. In the first and the second embodiments as described above, the filters Cy are used as the special filters and included in the filter unit of the color filter: however, in the fourth embodiment, a filter W that transmits light in the wavelength band for red, light in the wavelength band for green, and light in the wavelength band for blue is adopted as the special filter of the color filter. In the following, a configuration of the color filter according to the fourth embodiment is described, and thereafter, a method of reading an electrical signal from each of the pixels will be described. Meanwhile, the same components as those of the endoscope system 1 according to the first embodiment as described above are denoted by the same reference symbols, and detailed explanation thereof will be omitted.

Configuration of Color Filter

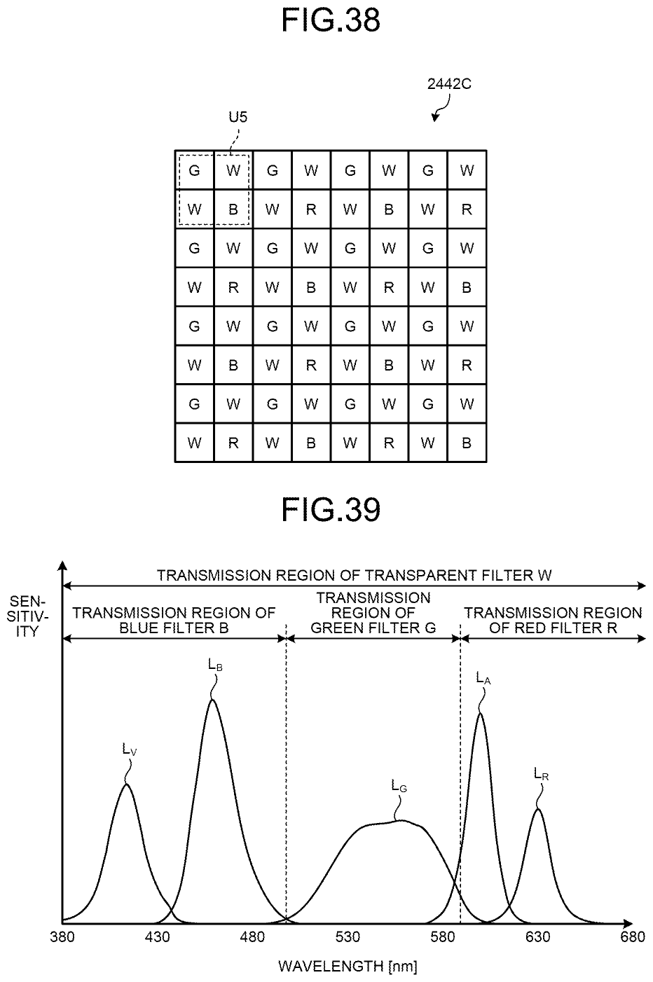

A color filter according to the fourth embodiment will be described in detail below. FIG. 38 is a diagram schematically illustrating arrangement of the color filter according to the fourth embodiment.

A color filter 2442C illustrated in FIG. 38 is configured such that unit pixels (2×2) constitute a single filter unit U5, and filters are arranged on the light receiving surfaces of the photoelectric conversion element PD11 to the photoelectric conversion element PD14. The filter unit U5 is configured with at least one of the blue filter B and the red filter R, the green filter G, and two or more special filters. The special filter is configured with a transparent filter W. The transparent filter W transmits light in the wavelength band for red, light in the wavelength band for green, and light in the wavelength band for blue.

FIG. 39 a diagram schematically illustrating sensitivity and a wavelength band of each of the filters. In FIG. 39, a horizontal axis represents a wavelength (nm) and a vertical axis represents sensitivity. Further, in FIG. 39, a curve L_{V} represents a wavelength band for purple, a curve L_{B} represents a wavelength band for blue, a curve L_{G} represents a wavelength band for green, a curve L_{A} represents a wavelength band for umber, and a curve L_{R} represents a wavelength band for red.

As illustrated in FIG. 39, the transparent filter W transmits light in the wavelength band for red, light in the wavelength band for green, and light in the wavelength band for blue. Meanwhile, in the following description, the photoelectric conversion element PD on which the transparent filter W is arranged is referred to as a W pixel.

Method of Reading Electrical Signal

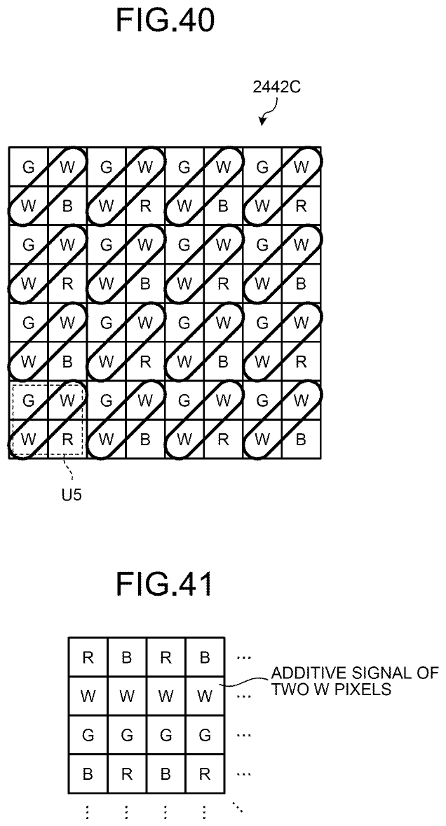

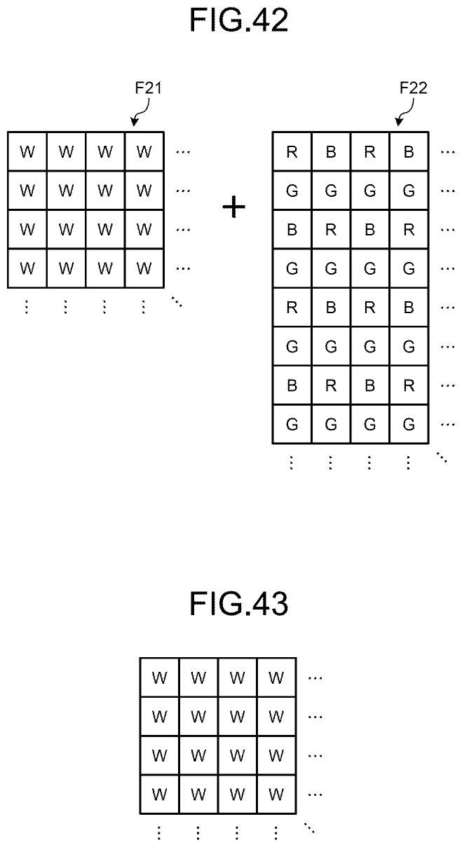

A method of reading electrical signals by the imaging control unit 2446 in the sensitivity-enhanced observation mode will be described below. FIG. 40 is a diagram schematically illustrating pixels added by the imaging control unit 2446 in the sensitivity-enhanced observation mode. FIG. 41 is a diagram schematically illustrating read of electrical signals from the image sensor 244 in the sensitivity-enhanced observation mode. FIG. 42 is a diagram schematically illustrating image frames that are output by the image sensor 244 in the sensitivity-enhanced observation mode.

As illustrated in FIG. 40 and FIG. 41, the imaging control unit 2446 causes the reading unit 2443 to apply a driving pulse to the transfer transistor Tr12 and the transfer transistor Tr13, so that charges are transferred from the photoelectric conversion element PD12 and the photoelectric conversion element PD13, in each of which the transparent filter W is arranged on the light receiving surface, to the charge-voltage converter FD1. Then, the imaging control unit 2446 causes the reading unit 2443 to output an additive signal that is obtained by addition performed by the charge-voltage converter FD1, for each of the filter units U5. Further, as illustrated in FIG. 42, the imaging control unit 2446 causes the pixel unit 2441 to alternately output a pixel mixed frame F21 in which the electrical signals generated by the plurality of W pixels are added, and a pixel non-mixed frame F22 that is generated by each of the R pixel, the G pixel, and the B pixel. As a result, because of wide wavelength transmission regions of the W pixels, the image sensor 244 is able to achieve sensitivity that is about four times higher than that of the R pixel, the G pixel, and the B pixel by adding the electrical signals of the two W pixels. Furthermore, the image sensor 244 reads and outputs the electrical signals from the R pixel, the G pixel, and the B pixel without adding the electrical signals, so that it is possible to increase a dynamic range by four times (12 dB). Meanwhile, a read timing in the sensitivity-enhanced observation mode is the same as that of the first embodiment as described above, and therefore, detailed explanation thereof will be omitted.

A method of reading electrical signals from the image sensor 244 in the high-speed observation mode will be described below. FIG. 43 is a diagram schematically illustrating read of electrical signals from the image sensor 244 in the high-speed observation mode.

As illustrated in FIG. 43, the imaging control unit 2446 causes the reading unit 2443 to apply a driving pulse to the transfer transistor Tr12 and the transfer transistor Tr13, so that charges are transferred from the photoelectric conversion element PD12 and the photoelectric conversion element PD13 to the charge-voltage converter FD1. Then, the imaging control unit 2446 causes the reading unit 2443 to output an additive signal that is obtained by addition performed by the charge-voltage converter FD1, for each of the filter units U5. In this case, the imaging control unit 2446 resets the electrical signals of pixels other than the plurality of W pixels, that is, the electrical signals of the R pixel, the G pixel, and the B pixel without reading the electrical signals. Meanwhile, a read timing in the high-speed observation mode is the same as that of the first embodiment as described above, and therefore, detailed explanation thereof will be omitted.

According to the fourth embodiment as described above, it is possible to reduce an accumulation time as compared to the normal observation, so that it is possible to perform imaging at high speed and it is possible to further reduce the size of the image sensor 244.

Furthermore, according to the fourth embodiment, in the special observation mode, the imaging control unit 2446 adds, for each of the filter units U5, the electrical signals generated by the plurality of W pixels on which the special filters are arranged, and sequentially outputs the additive signals to outside. Therefore, it is possible to increase sensitivity due to pixel mixture and reduce an accumulation time as compared to the normal observation mode. As a result, it is possible to perform imaging at high speed and it is possible to further reduce the size of the image sensor 244.

Moreover, according to the fourth embodiment, in the high-speed observation mode, the imaging control unit 2446 performs discharges without outputting the electrical signals from the R pixel, the G pixel, and the B pixel and resets the R pixel, the G pixel, and the B pixel, so that it is possible to further reduce a read interval as compared to the sensitivity-enhanced observation mode.

Furthermore, according to the fourth embodiment, because of wide wavelength transmission regions of the W pixels, the imaging control unit 2446 is able to achieve sensitivity that is about four times higher than that of the R pixel, the G pixel, and the B pixel by adding the electrical signals of the two W pixels, and reads and outputs the electrical signals from the R pixel, the G pixel, and the B pixel without adding the electrical signals, so that it is possible to increase a dynamic range by four times (12 dB).

Moreover, according to the fourth embodiment, in the special observation mode, the imaging control unit 2446 adds, for each of the filter units U5, the electrical signals generated by the plurality of W pixels on which the special filters are arranged, sequentially outputs the additive signals to outside, and sequentially outputs the electrical signals generated by the R pixel, the G pixel, and the B pixel. Therefore, it is possible to increase sensitivity due to synthesis with a non-mixed electrical signal, and simultaneously ensure color resolution.

Furthermore, according to the fourth embodiment, in the special observation mode, the imaging control unit 2446 adds, for each of the filter units U5, the electrical signals generated by the plurality of W pixels on which the special filters are arranged, and sequentially outputs the additive signals to outside. Therefore, it becomes not necessary to perform imaging twice for long-time exposure and short-time exposure to increase sensitivity, and it is possible to prevent generation of an artifact due to motion.

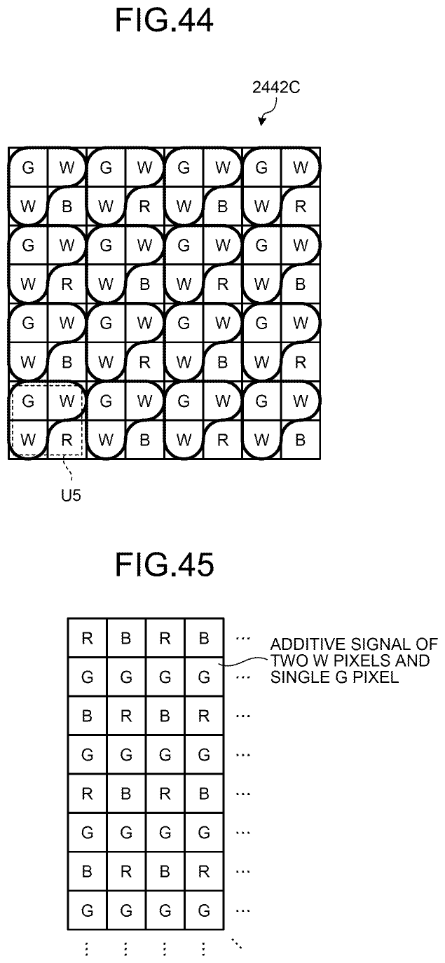

Meanwhile, in the fourth embodiment, similarly to the first modification of the first embodiment as described above, as illustrated in FIG. 44 and FIG. 45, the imaging control unit 2446 may control the reading unit 2443 and perform the same process as that of the first modification of the first embodiment as described above such that the electrical signals of the two W pixels and the electrical signal of the G pixel are added and output to outside for each of the filter units U5.

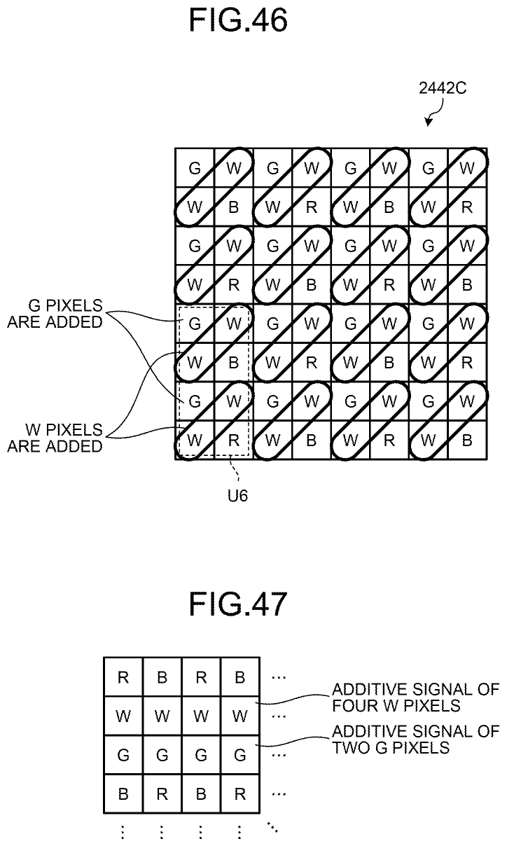

Furthermore, in the fourth embodiment, if the pixel unit 2441A of the second embodiment as described above is used, as illustrated in FIG. 46 and FIG. 47, the imaging control unit 2446 may control the reading unit 2443 and preform the same process as that of the second embodiment as described above to output an additive signal in which the electrical signals of the four W pixels are added for each of filter units U6.

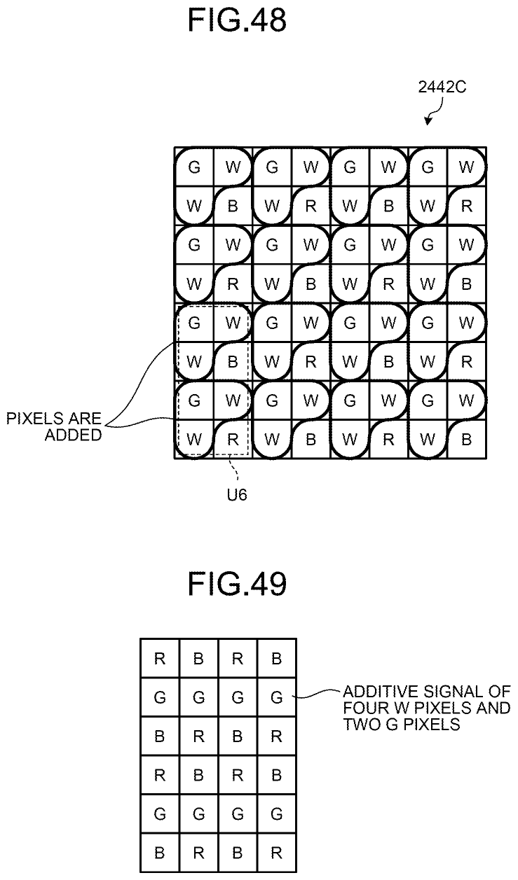

Moreover, in the fourth embodiment, if the pixel unit 2441A of the second embodiment as described above is used, as illustrated in FIG. 48 and FIG. 49, the imaging control unit 2446 may control the reading unit 2443 and perform the same process as that of the first modification of the second embodiment as described above to output an additive signal in which the electrical signals of the four W pixels and the electrical signals of the two G pixels are added by the charge-voltage converter FD1 for each of the filter units U6.

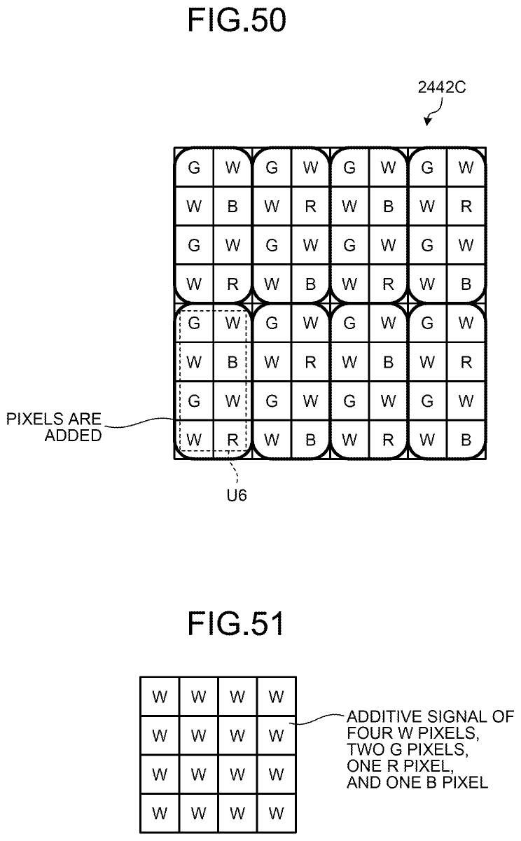

Furthermore, in the fourth embodiment, if the pixel unit 2441A of the second embodiment as described above is used, as illustrated in FIG. 50 and FIG. 51, the imaging control unit 2446 may control the reading unit 2443 and perform the same process as that of the second modification of the second embodiment as described above to output an additive signal in which the electrical signals of the four W pixels, the electrical signals of the two G pixels, the electrical signal of the R pixel, and the electrical signal of the B pixel are added by the charge-voltage converter FD1 for each of the filter units U6.

### Other Embodiments

In the first to the fourth embodiments as described above, the imaging control unit 2446 adds the electrical signals of the plurality of Cy pixels and outputs the additive signal to outside in the special observation mode, but the A/D converter 2444 may output a digital signal in which a bit depth is reduced to below a predetermined number of bits in an A/D conversion process performed by the A/D converter 2444. For example, the imaging control unit 2446 may reduce the number of bits to be converted by the A/D converter 2444 from the predetermined number of bits (10) to N to reduce a time of the A/D conversion process from 2¹⁰ to 2^{N} and to reduce a transmission time accordingly. It is of course possible to perform a process of reducing the number of bits in addition to the process performed in the first to the fourth embodiments as described above. With this configuration, it is possible to further increase processing speed.

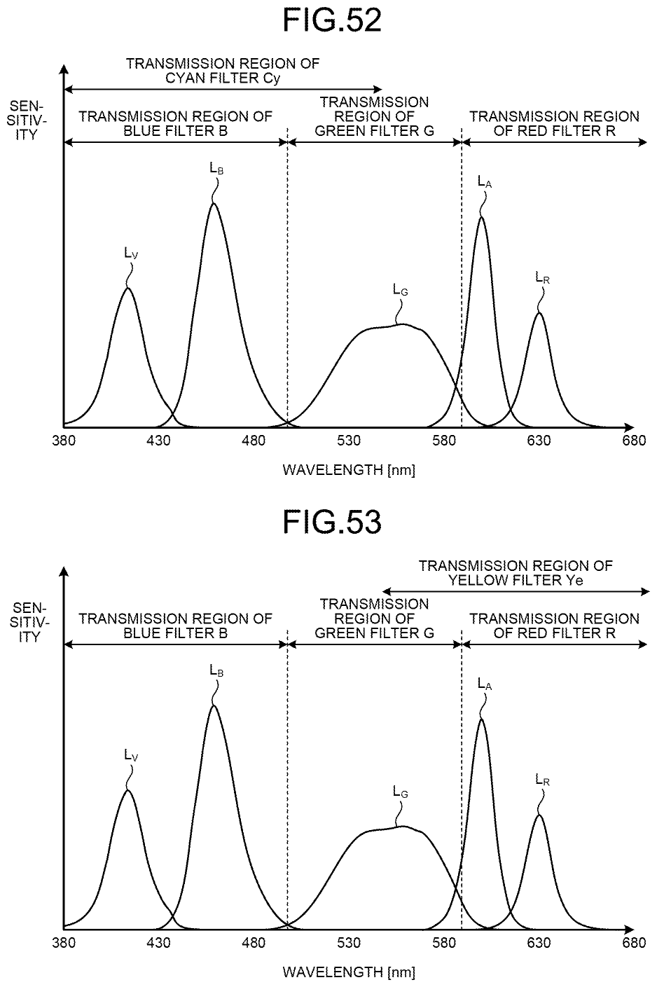

Furthermore, in the first and the second embodiments, the cyan filter Cy transmits light in the wavelength band for blue and light in the wavelength band for green, but it is sufficient to transmit a part of light in the wavelength band for green as illustrated in FIG. 52. It is of course possible that the cyan filter Cy transmits a part of light in the wavelength band for blue and light in the wavelength band for green. In addition, the cyan filter Cy may transmit a part of light in the wavelength band for blue and a part of light in the wavelength band for green.

Moreover, in the third embodiment, the yellow filter Ye transmits light in the wavelength band for red and light in the wavelength band for green, but it is sufficient to transmit a part of light in the wavelength band for green as illustrated in FIG. 53. It is of course possible that the yellow filter Ye transmits a part of light in the wavelength band for red and light in the wavelength band for green. Moreover, the yellow filter Ye may transmit a part of light in the wavelength band for red and a part of light in the wavelength band for green.

Furthermore, various modes may be made by appropriately combining a plurality of components disclosed in the endoscope system according to the first to the fourth embodiments. For example, some components may be removed from all of the components of the endoscope system descried in the first to the fourth embodiments.

Moreover, in the first to the fourth embodiments as described above, “units” described above may be replaced with “means”, “circuits”, or the like. For example, the control unit may be replaced with a control means or a control circuit.

Furthermore, the programs to be executed by the endoscope system according to the first to the fourth embodiments are provided by being recorded, as file data in an installable format or an executable format, on a computer readable recording medium such as a compact disc read only memory (CD-ROM), a flexible disk (FD), a compact disk recordable (CD-R), a digital versatile disk (DVD), a USB medium, or a flash memory.

Moreover, the programs to be executed by the endoscope system according to the first to the fourth embodiments may be stored on a computer connected to a network, such as the Internet, and provided by causing the programs to be downloaded via the network.

In describing the flowcharts in this specification, context of the processes among the steps is disclosed by using expressions such as “first”, “thereafter”, and “subsequently”, but the sequences of the processes needed for carrying out the present disclosure are not uniquely defined by these expressions. In other words, the sequences of the processes in the flowcharts described in the present specification may be modified as long as there is no contradiction.

According to the present disclosure, it is possible to perform imaging at high speed and reduce a size of a device.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image sensor comprising:
a pixel unit including a plurality of pixels that are arranged in a two-dimensional matrix manner, each pixel being configured to perform photoelectric conversion and generate an electrical signal corresponding to intensity of received light;
a color filter including a plurality of filter units, each filter unit including at least one of a blue filter and a red filter, a green filter, and two or more of special filters, the plurality of filter units being arranged on the plurality of pixels such that each of filters included in the filter unit corresponds to each of predetermined pixels of the plurality of pixels, the blue filter transmitting light in a wavelength band for blue, the red filter transmitting light in a wavelength band for red, the green filter transmitting light in a wavelength band for green, each of the two or more of special filters being one of a cyan filter and a yellow filter, the cyan filter transmitting light in the wavelength band for blue and light in the wavelength band for green, the yellow filter transmitting light in the wavelength band for green and light in the wavelength band for red; and
an imaging controller configured to
in a normal observation mode, sequentially output electrical signals generated by the plurality of pixels, and
in a special observation mode, sequentially output an additive signal in which electrical signals generated by the plurality of pixels on which at least the two or more of special filters are arranged are added for each of the filter units.

2. The image sensor according to claim 1, wherein in the special observation mode, the imaging controller is configured to output an additive signal in which the electrical signals generated by the plurality of pixels on which at least the two or more of special filters are arranged and the electrical signal generated by the pixel on which the green filter is arranged are added for each of the filter units.

3. The image sensor according to claim 1, wherein
each of the filter units includes at least two green filters and four special filters, and
in the special observation mode, the imaging controller is configured to
output an additive signal in which the electrical signals generated by the plurality of pixels on which the four special filters are arranged are added for each of the filter units, and
output an additive signal in which the electrical signals generated by the plurality of pixels on which the two green filters are arranged are added.

4. The image sensor according to claim 1, wherein
each of filter units includes at least two green filters and the four special filters, and
in the special observation mode, the imaging controller is configured to
output an additive signal in which the electrical signals generated by the plurality of pixels on which the four special filters are arranged and the electrical signals generated by the plurality of pixels on which the two green filters are arranged are added for each of the filter units.

5. The image sensor according to claim 1, wherein in the special observation mode, the imaging controller is configured to output an additive signal in which the electrical signals generated by the plurality of pixels are added for each of the filter units.

6. The image sensor according to claim 1, wherein
in the special observation mode, the imaging controller is configured to cause the pixel unit to alternately output a pixel mixed frame and a pixel non-mixed frame,
the pixel mixed frame is a frame in which the electrical signals generated by at least the plurality of pixels on which the two or more of special filters are arranged are added, and
the pixel non-mixed frame is a frame that includes the electrical signal generated by the pixel on which at least one of the blue filter and the red filter is arranged and includes the electrical signal generated by the pixel on which the green filter is arranged.

7. The image sensor according to claim 1, wherein in the special observation mode, the imaging controller is configured to
output a pixel mixed frame in which the electrical signals generated by at least the plurality of pixels on which the two or more of special filters are arranged are added, and
reset a signal charge that are accumulated by the pixel on which at least one of the blue filter and the red filter is arranged and a signal charge that are accumulated by the pixel on which the green filter is arranged, every time the pixel mixed frame is output.

8. The image sensor according to claim 1, further comprising:
an analog-to-digital (A/D) converter, wherein
the A/D converter is configured to
perform an A/D conversion process to change the electrical signal input from the pixel unit to a digital signal with a predetermined number of bits, and
output the digital signal, and
the imaging controller is configured to, in the special observation mode, cause the A/D converter to output a digital signal for which a bit depth is reduced to below the predetermined number of bits in the A/D conversion process.

9. The image sensor according to claim 1, wherein each of the two or more of special filter is a transparent filter transmitting light in the wavelength band for red, light in the wavelength band for green, and light in the wavelength band for blue.

10. An endoscope comprising:
the image sensor according to claim 1; and
an insertion portion, wherein
the insertion portion includes a distal end portion that is insertable into a subject, and
the image sensor is arranged on the distal end portion.

11. An endoscope system comprising:
the endoscope according to claim 10;
a light source configured to apply illumination light to the endoscope, the illumination light including at least one of light in the wavelength band for blue and light in the wavelength band for red and including light in the wavelength band for green; and
a control device configured to generate a display image based on a digital signal input from the image sensor.
